(19) United States
(12) Patent Application Publication
(10) Pub. No.: US 2024/0302384 A1
(43) Pub. Date: Sep. 12, 2024

(54) METABOLOMIC SIGNATURE FOR THE DIAGNOSIS OF ACUTE MESENTERIC ISCHEMIA

(71) Applicants: APHP (ASSISTANCE PUBLIQUE - HÔPITAUX DE PARIS ), Paris (FR); UNIVERSITE PARIS, Paris (FR); INSERM (INSTITUT NATIONAL DE LA SANTÉ ET DE LA RECHERCHE MÉDICALE ), Paris Cedex 13 (FR); IMPERIAL COLLEGE OF SCIENCE, TECHNOLOGY AND MEDICINE, London (GB)

(72) Inventors: Yoram BOUHNIK, Clichy (FR); Alexandre NUZZO, Clichy (FR); Olivier CORCOS, Clichy (FR); Marc-Emmanuel DUMAS, London (GB); Antonis MYRIDAKIS, London (GB); Dominique GAUGUIER, Paris (FR)

(73) Assignees: APHP (ASSISTANCE PUBLIQUE - HÔPITAUX DE PARIS ), Paris (FR); UNIVERSITE PARIS CITÉ, Paris (FR); INSERM (INSTITUT NATIONAL DE LA SANTÉ ET DE LA RECHERCHE MÉDICALE ), Paris Cedex 13 (FR); IMPERIAL COLLEGE OF SCIENCE, TECHNOLOGY AND MEDICINE, London (GB)

(21) Appl. No.: 18/547,759

(22) PCT Filed: Mar. 3, 2022
(86) PCT No.: PCT/EP2022/055463

§ 371 Date: Aug. 24, 2023

(30) Foreign Application Priority Data
Mar. 3, 2021 (EP) 21305252.5

(51) Int. Cl. G01N 33/68; G01N 33/53; G01N 33/92

(52) U.S. Cl. CPC G01N 33/6893; G01N 33/5308; G01N 33/92; G01N 2570/00; G01N 2800/06; G01N 2800/7019

(57) ABSTRACT

A metabolomic signature of acute mesenteric ischemia (AMI) and the determination thereof in a method for identifying a subject suffering or being at risk of suffering from AMI. Also, a kit that includes elements for determining the metabolomic signature of AMI and implementing the method for identifying a subject suffering or being at risk of suffering from AMI.

### FIELD OF INVENTION

The present invention relates to the diagnosis of acute mesenteric ischemia (AMI), and in particular to a metabolomic signature of AMI and the determination thereof in a method for identifying a subject suffering or being at risk of suffering from AMI.

### BACKGROUND OF INVENTION

Acute mesenteric ischemia (AMI) is caused by an inadequate blood flow through the mesenteric vessels (either arterial or venous), resulting in ischemia, inflammatory injury and cellular damage. If left untreated, AMI may lead to tissue necrosis, requiring intestinal resection, and ultimately to patient death.

AMI accounts for about 0.1% of hospital admissions and is associated with a mortality rate of 60% to 80%. Mortality and intestinal resection rates have remained unchanged for decades despite the progresses made in radiology, endovascular procedures, and intensive care medicine. However, recent reports suggest improved outcomes for patients if diagnosis and standardized multidisciplinary expert care are provided at an early stage (Corcos et al., Clin Gastroenterol Hepatol. 2013 February; 11(2):158-65.e2; Ding et al., J Clin Gastroenterol. 2017 October; 51(9):e77-e82; Nuzzo et al., Am J Gastroenterol. 2019 February; 114(2):348-351). Indeed, early AMI is a fully reversible condition, as opposed to advanced AMI with irreversible transmural necrosis (Clair et al., N Engl J Med. 2016 Mar. 10; 374(10):959-68).

Early management of AMI can thus avoid fatal outcome and prevent lifelong complications or impairments such as short bowel syndrome. However, AMI patients generally present unspecific acute abdominal pain which renders clinical suspicion and identification challenging, and can often lead to missed or delayed diagnosis and care. Moreover, when the suspicion is not evoked, AMI may be underdiagnosed on contrast-enhanced computed tomography (CT) scan of the acute abdomen (Lehtimaki et al., Eur J Radiol. 2015 December; 84(12):2444-53).

A number of studies have sought to identify biomarkers specifically associated with AMI, with the aim of developing a reliable AMI diagnosis test for patients presenting with acute abdominal pain. While biologic abnormalities—such as leukocytosis or lactic acidosis—have been documented in patients with AMI, their performance to establish an early diagnosis is poor (Peoc'h et al., Clin Chem Lab Med. 2018 Feb. 23; 56(3):373-385; Derikx et al., Best Pract Res Clin Gastroenterol. 2017 February; 31(1):69-74). The high complexity of the layered intestinal wall structure increases the diversity of the proteins and metabolites released in AMI. Their hepatic metabolism through the hepatic portal system results in substantial overlap with liver proteins and metabolites. These factors, along with the heterogeneity of the disease, explain why identifying clinically reliable biomarkers of AMI has remained unsuccessful so far.

Therefore, there is still a dire need for biomarkers that could be reliably and easily assessed to diagnose AMI in patients presenting with acute abdominal pain. Indeed, as mentioned above, timely diagnosis of AMI is critical to ensure immediate and appropriate care is provided and thus avoid death or lifelong complications or impairments such as short bowel syndrome.

Following the results of a pilot study showing an improvement in survival and lower resection rates (Corcos et al., Clin Gastroenterol Hepatol. 2013 February; 11(2):158-65.e2), the Inventors created an intestinal stroke center (Beaujon Hospital, Clichy, France) that provides 24/7 standardized multimodal and multidisciplinary care to AMI patients referred from all hospitals in the Paris region. Since the creation of this center in 2016, the Inventors have prospectively enrolled patients who undergo a contrast-enhanced CT scan for acute abdominal pain as part of the SURVIBIO diagnostic study. Blood samples are collected on admission and stored in a biobank for further biomarker analysis and research. As detailed herein, the Inventors have used the admission plasma samples from the biobank to identify novel biomarkers for diagnosis of AMI using untargeted GC-MS and ¹H-NMR metabolomics.

The present invention thus relates to a metabolomic signature of acute mesenteric ischemia (AMI) and the determination thereof in a method for identifying a subject suffering or being at risk of suffering from AMI. The present invention also relates to a kit comprising means for determining the metabolomic signature of AMI and implementing the method for identifying a subject suffering or being at risk of suffering from AMI.

### SUMMARY

The present invention relates to a method for identifying a subject suffering or being at risk of suffering from acute mesenteric ischemia (AMI), said method comprising:
a) determining a metabolomic signature of the subject by measuring in a biological sample previously obtained from the subject the level, amount, or concentration of at least one biomarker selected from the group consisting of isomaltose, glutamine, phenylalanine, glycerol, L1PN, 1-monoolein, L1AB, L3TG, threose, H4A2, L1TG, TPA2, and L1PL;
b) comparing the metabolomic signature of the subject to a reference metabolomic signature; and
c) identifying the subject suffering or being at risk of suffering from AMI based on the comparison of his/her metabolomic signature to the reference metabolomic signature.

The present invention also relates to method for providing an adapted care to a subject identified as suffering or being at risk of suffering from acute mesenteric ischemia (AMI), said method comprising:
a) determining a metabolomic signature of the subject by measuring in a biological sample previously obtained from the subject the level, amount, or concentration of at least one biomarker selected from the group consisting of isomaltose, glutamine, phenylalanine, glycerol, L1PN, 1-monoolein, L1AB, L3TG, threose, H4A2, L1TG, TPA2, and L1PL;
b) comparing the metabolomic signature of the subject to a reference metabolomic signature; and
c) providing an adapted care to the subject identified as suffering or being at risk of suffering from AMI based on the comparison of his/her metabolomic signature to the reference metabolomic signature.

In one embodiment, the metabolomic signature is determined by measuring in a biological sample previously obtained from the subject the level, amount, or concentration of at least one biomarker selected from the group consisting of isomaltose, glutamine, phenylalanine, glycerol, L1PN, and 1-monoolein. In one embodiment, the metabolomic signature is determined by measuring in a biological sample previously obtained from the subject the level, amount, or concentration of isomaltose, glutamine, phenylalanine, glycerol, and L1PN. In one embodiment, the metabolomic signature is determined by measuring in a biological sample previously obtained from the subject the level, amount, or concentration of isomaltose, glutamine, phenylalanine, glycerol, L1PN, 1-monoolein, L1AB, L3TG, threose, H4A2, L1TG, TPA2, and L1PL.

In one embodiment, the reference metabolomic signature is derived from the measure of the level, amount, or concentration of the same at least one biomarker in biological samples previously obtained from a population of subjects suffering from non-ischemic abdominal pain.

In one embodiment, the biological sample previously obtained from the subject is a blood sample, preferably a plasma sample.

The present invention also relates to a kit for implementing the methods of the invention, comprising:
means for measuring the level, amount, or concentration of at least one biomarker selected from the group consisting of isomaltose, glutamine, phenylalanine, glycerol, L1PN, 1-monoolein, L1AB, L3TG, threose, H4A2, L1TG, TPA2, and L1PL;
optionally internal standard(s) and/or control(s); and
optionally instructions for use.

In one embodiment, the kit comprises means for measuring the level, amount, or concentration of at least one biomarker selected from the group consisting of isomaltose, glutamine, phenylalanine, glycerol, L1PN, and 1-monoolein. In one embodiment, the kit comprises means for measuring the level, amount, or concentration of isomaltose, glutamine, phenylalanine, glycerol, and L1PN. In one embodiment, the kit comprises means for measuring the level, amount, or concentration of isomaltose, glutamine, phenylalanine, glycerol, L1PN, 1-monoolein, L1AB, L3TG, threose, H4A2, L1TG, TPA2, and L1PL.

The present invention also relates to a metabolomic signature of acute mesenteric ischemia (AMI), said metabolomic signature comprising at least one biomarker selected from the group consisting of isomaltose, glutamine, phenylalanine, glycerol, L1PN, 1-monoolein, L1AB, L3TG, threose, H4A2, L1TG, TPA2, and L1PL.

In one embodiment, the metabolomic signature comprises at least one biomarker selected from the group consisting of isomaltose, glutamine, phenylalanine, glycerol, L1PN, and 1-monoolein. In one embodiment, the metabolomic signature further comprises glycerol. In one embodiment, the metabolomic signature comprises isomaltose, glutamine, phenylalanine, glycerol, and L1PN. In one embodiment, the metabolomic signature further comprises glycerol. In one embodiment, the metabolomic signature comprises 1-monoolein, isomaltose, phenylalanine, L1PN, L1AB, L3TG, threose, H4A2, L1TG, TPA2, L1PL, glutamine, and glycerol.

### Definitions

In the present invention, the following terms have the following meanings:

The terms “a” and “an” refer to one or to more than one (i.e., to at least one) of the grammatical object of the article. By way of example, “a biomarker” means one biomarker or more than one biomarker.

“About”, preceding a figure encompasses plus or minus 10%, or less, of the value of said figure. It is to be understood that the value to which the term “about” refers is itself also specifically, and preferably, disclosed.

“AMI” refers to acute mesenteric ischemia, which is caused by an inadequate blood flow through the mesenteric vessels (either arterial or venous), that may result in ischemia, inflammatory injury and cellular damage. AMI may also be known as intestinal ischemia or acute intestinal ischemic injury (or i3) or ischemic enteritis or mesenteric infarction. In one embodiment, AMI is defined as the presence of i) acute clinical, biological and/or contrast-enhanced CT scan features of bowel injury; ii) vascular insufficiency (occlusive or non-occlusive) of the celiac trunk and/or the superior mesentery artery and/or superior mesenteric vein; and/or iii) the absence of an alternative diagnosis.

“AUROC” stands for area under the ROC curve (and is also known as area under the curve or AUC), and is an indicator of the accuracy of a diagnostic method or test. In statistics, a receiver operating characteristic (ROC), or ROC curve, is a graphical plot that illustrates the performance of a binary classifier system as its discrimination threshold is varied. The curve is created by plotting the sensitivity against the specificity (usually 1-specificity) at successive values from 0 to 1. ROC curves and AUC (or AUROC) are well-known in the field of statistics. Usually, it is considered that an AUC or AUROC between 0.5 and 0.7 indicates that the diagnostic method or test has poor to fair discrimination. By contrast, AUROC between 0.7 and 0.8 indicates that the diagnostic method or test has satisfactory discrimination and AUROC between 0.8 and 0.9 indicates that the diagnostic method or test has excellent discrimination (Forthofer et al. (2006) Biostatistics: A guide to Design, Analysis, and Discovery: Elsevier).

“Biomarker” or “biological marker” refers to a variable, such as a metabolite, that can be measured in a biological sample from a subject.

“Measuring” or “measurement”, or alternatively “detecting” or “detection”, mean assessing the presence, absence, level, quantity, or amount of a given substance, e.g., a biomarker. “Measuring” or “measurement”, or alternatively “detecting” or “detection” as used herein include the derivation of the qualitative or quantitative concentration of said substance, in particular said biomarker.

“Non-invasive”, when referring to a method or test according to the present invention, means that the method or test of the invention does not comprise obtaining or recovering a tissue sample from the body of a subject. In one embodiment, a blood sample is not considered as a tissue sample.

“Diagnostic target” refers to the main objective of a diagnostic method or test. For example, a diagnostic method wherein the diagnostic target is AMI aims at assessing the presence of AMI in a subject.

“Sensitivity”, for a diagnostic method, measures the proportion of patients with the diagnostic target (e.g., AMI) that are correctly identified as such by the diagnostic method.

“Specificity”, for a diagnostic method, measures the proportion of patients without the diagnostic target (e.g., AMI) that are correctly identified as such by the diagnostic method.

“Subject” refers to a mammal, preferably to a primate, more preferably to a human.

“Treating” or “treatment” refers to a therapeutic treatment, to a prophylactic (or preventative) treatment, or to both a therapeutic treatment and a prophylactic (or preventative) treatment; wherein the object is to prevent or slow down or lessen acute mesenteric ischemia (AMI), its associated symptoms and/or complications such as ischemia, inflammatory injury, cellular damage, tissue necrosis and/or intestinal resection. In one embodiment, a subject is successfully “treated” if, after receiving treatment as described herein, including adapted care as described herein, the subject shows at least one of the following: relief to some extent of one or more of the symptoms and/or complications associated with AMI, and/or improvement in quality-of-life issues. The above parameters for assessing successful treatment and improvement in the symptoms and/or complications associated with AMI are readily measurable by routine procedures familiar to a physician.

### DETAILED DESCRIPTION

The present invention relates to a metabolomic signature or metabolomic profile of acute mesenteric ischemia (AMI). As used herein, the terms “metabolomic signature” and “metabolomic profile” are interchangeable.

According to one embodiment, said metabolomic signature is a metabolomic signature of early AMI.

As used herein, early AMI corresponds to a reversible stage of AMI. Timely treated, patients suffering from early AMI can thus make a full recovery and in particular recover normal digestive function.

In one embodiment, early AMI is characterized by the absence of intestinal necrosis. In one embodiment, early AMI is characterized by the absence of organ failure, the absence of increased blood lactate, and/or the absence of pneumoperitoneum/peritonitis on CT scan.

According to one embodiment, the metabolomic signature of AMI comprises or consists of biomarker(s) whose presence or absence is characteristic of AMI.

In one embodiment, the metabolomic signature of AMI comprises or consists of biomarker(s) whose presence or absence is characteristic of AMI when compared to a reference metabolomic signature. Accordingly, in one embodiment, “is/are/being characteristic of AMI”, when referring to biomarker(s), means that said biomarker(s) is/are specifically present in a subject suffering from AMI, that is to say said biomarker(s) is/are absent in a reference subject not suffering from AMI or in a reference population of subjects who are not suffering from AMI. In one embodiment, “is/are/being characteristic of AMI”, when referring to biomarker(s), means that said biomarker(s) is/are specifically absent in a subject suffering from AMI, that is to say said biomarker(s) is/are present in a reference subject not suffering from AMI or in a reference population of subjects who are not suffering from AMI.

According to one embodiment, the metabolomic signature of AMI comprises or consists of biomarker(s) whose level, amount, or concentration is characteristic of AMI.

In one embodiment, the metabolomic signature of AMI comprises or consists of biomarker(s) whose level, amount, or concentration is characteristic of AMI when compared to a reference metabolomic signature. Accordingly, in one embodiment, “is/are/being characteristic of AMI”, when referring to the level, amount, or concentration of biomarker(s), means that the level, amount, or concentration of a given biomarker is substantially different from or substantially similar to the level, amount, or concentration of the same biomarker measured in a reference subject or derived from a reference population of subjects. Whether “characteristic” means being “substantially different” or “substantially similar” depends on the reference subject (or the reference population of subjects) and their disease status.

In one embodiment, the reference subject is a subject suffering from non-ischemic abdominal pain (i.e., abdominal pain not caused by mesenteric ischemia), or the population of subjects is a population of subjects suffering from non-ischemic abdominal pain. Accordingly, in said embodiment, “being characteristic of AMI” means being “substantially different”.

In one embodiment, the reference subject is a substantially healthy subject, or the population of subjects is a population of substantially healthy subjects. Accordingly, in said embodiment, “being characteristic of AMI” means being “substantially different”.

In one embodiment, the reference subject is a subject suffering from AMI or the population of subjects is a population of subjects suffering from AMI. Accordingly, in said embodiment, “being characteristic of AMI” means being “substantially similar”.

In one embodiment, the level, amount, or concentration of a given biomarker is “substantially different” if it is more than about 1% higher, 2% higher, 3% higher, 4% higher, 5% higher, 6% higher, 7% higher, 8% higher, 9% higher, 10% higher, 15% higher, 20% higher, 25% higher, 30% higher, 35% higher, 40% higher, 45% higher, or 50% higher, or more than the level, amount, or concentration of the same biomarker measured in a reference subject or derived from a reference population of subjects; or if it is more than about 1% lower, 2% lower, 3% lower, 4% lower, 5% lower, 6% lower, 7% lower, 8% lower, 9% lower, 10% lower, 15% lower, 20% lower, 25% lower, 30% lower, 35% lower, 40% lower, 45% lower, or 50% lower, or more than the level, amount, or concentration of the same biomarker measured in a reference subject or derived from a reference population of subjects. In one embodiment, the level, amount, or concentration of a given biomarker is “substantially different” if it is more than about 5% higher or 5% lower than the level, amount, or concentration of the same biomarker measured in a reference subject or derived from a reference population of subjects.

In one embodiment, the level, amount, or concentration of a given biomarker is “substantially different” if the given biomarker is present in a subject suffering from AMI and absent in a reference subject not suffering from AMI or in a reference population of subjects who are not suffering from AMI. In one embodiment, the level, amount, or concentration of a given biomarker is “substantially different” if the given biomarker is absent in a subject suffering from AMI and present in a reference subject not suffering from AMI or in a reference population of subjects who are not suffering from AMI.

In one embodiment, the level, amount, or concentration of a given biomarker is “substantially similar” if it is less than about 1% higher, 2% higher, 3% higher, 4% higher, 5% higher, 6% higher, 7% higher, 8% higher, 9% higher, 10% higher, 15% higher, or 20% higher than the level, amount, or concentration of the same biomarker measured in a reference subject or derived from a reference population of subjects; or if it is less than about 1% lower, 2% lower, 3% lower, 4% lower, 5% lower, 6% lower, 7% lower, 8% lower, 9% lower, 10% lower, 15% lower, or 20% lower than the level, amount, or concentration of the same biomarker measured in a reference subject or derived from a reference population of subjects. In one embodiment, the level, amount, or concentration of a given biomarker is “substantially similar” if it is less than about 5% higher or 5% lower than the level, amount, or concentration of the same biomarker measured in a reference subject or derived from a reference population of subjects.

In one embodiment, the level, amount, or concentration of biomarker(s) may be measured by methods well-known in the art. Such methods include for example mass spectrometry, including chromatography-assisted mass spectrometry (such as gas chromatography-mass spectrometry or GC-MS, and liquid chromatography-mass spectrometry or LC-MS) and hydrogen-1 nuclear magnetic resonance spectroscopy also known as proton nuclear magnetic resonance spectrometry (¹H-NMR); chromatography, including high-performance liquid chromatography (HPLC) and high-performance anion-exchange chromatography with pulsed amperometric detection (HPAEC-PAD); immunohistochemistry; immunoassays; enzyme-linked immunosorbent assay (ELISA); western blots; enzymatic methods.

In one embodiment, the metabolomic signature of AMI comprises at least 1 or consists of 1 biomarker selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol.

According to the present invention, the metabolomic signature of AMI may thus comprise or consist of a biomarker being a lipid (such as 1-monoolein); a biomarker being a sugar alcohol also known as polyhydric alcohol or polyol (such as isomaltose, threose, melibiose, D-allose, quinic acid, and/or glycerol); a biomarker being an amino acid (such as phenylalanine and/or glutamine); a biomarker being a vitamin (such as alpha-tocopherol); a biomarker being an organic acid (such as 2-hydroxybutyric acid); a biomarker being a fatty acid (such as 2-ethylhexanoic acid, palmitic acid, succinic acid, and/or oleic acid); a biomarker being a sugar acid (such as threonic acid and/or glyceric acid); a biomarker being an alpha-keto acid (such as pyruvic acid and/or 2-ketoisocaproic acid); a biomarker being an inorganic compound (such as phosphoric acid); a biomarker being a sterol (such as cholesterol); a biomarker being a carboxylic acid (such as acetic acid); and/or a biomarker being a lipoprotein subfractions (such as low density lipoprotein (LDL) subfractions, for example L1PN, L1AB, L3TG, L1TG, L1PL, L6FC, L5PL, L5CH, L5FC, L5AB, L5PN, and/or L6PL; high density lipoprotein (HDL) subfractions, for example H4A2, H4PL, HDA2, H4A1, H4CH, H3FC, H4FC, HDA1, H3A2, and/or H3PL; apolipoprotein A1 (Apo-A1) subfractions, for example TPA1; and/or apolipoprotein A2 (Apo-Ab) subfractions, for example TPA2).

In one embodiment, the metabolomic signature of AMI does not comprise or consist of glycerol. In one embodiment, the metabolomic signature of AMI does not comprise or consist of glutamine. In one embodiment, the metabolomic signature of AMI does not comprise or consist of glycerol and does not comprise or consist of glutamine.

In one embodiment, the metabolomic signature of AMI comprises at least 1 or consists of 1 biomarker selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, and glutamine; and optionally further comprises glycerol. In one embodiment, the metabolomic signature of AMI comprises at least 1 or consists of 1 biomarker selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid; and optionally further comprises glycerol and/or glutamine.

In one embodiment, the metabolomic signature of AMI comprises at least 2, 3, 4, 5, 6, 7, 8, 9, 10, 11, 12, 13, or more biomarkers selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol. In one embodiment, the metabolomic signature of AMI comprises or consists of 2, 3, 4, 5, 6, 7, 8, 9, 10, 11, 12, 13, or more biomarkers selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol.

In one embodiment, the metabolomic signature of AMI comprises at least 2, 3, 4, 5, 6, 7, 8, 9, 10, 11, 12, 13, or more biomarkers selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, and glutamine; and optionally further comprises glycerol. In one embodiment, the metabolomic signature of AMI comprises or consists of 2, 3, 4, 5, 6, 7, 8, 9, 10, 11, 12, 13, or more biomarkers selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, and glutamine; and optionally further comprises glycerol.

In one embodiment, the metabolomic signature of AMI comprises at least 1 or consists of 1 biomarker selected from the group comprising or consisting of 1-monoolein, isomaltose, phenylalanine, L1PN, L1AB, L3TG, threose, H4A2, L1TG, TPA2, L1PL, glutamine, and glycerol. In one embodiment, the metabolomic signature of AMI comprises at least 1 or consists of 1 biomarker selected from the group comprising or consisting of 1-monoolein, isomaltose, phenylalanine, L1PN, L1AB, L3TG, threose, H4A2, L1TG, TPA2, L1PL, and glutamine; and optionally further comprises glycerol. In one embodiment, the metabolomic signature of AMI comprises at least 1 or consists of 1 biomarker selected from the group comprising or consisting of 1-monoolein, isomaltose, phenylalanine, L1PN, L1AB, L3TG, threose, H4A2, L1TG, TPA2, and L1PL; and optionally further comprises glycerol and/or glutamine.

In one embodiment, the metabolomic signature of AMI comprises at least 2, 3, 4, 5, 6, 7, 8, 9, 10, 11, or 12 biomarkers selected from the group comprising or consisting of 1-monoolein, isomaltose, phenylalanine, L1PN, L1AB, L3TG, threose, H4A2, L1TG, TPA2, L1PL, glutamine, and glycerol. In one embodiment, the metabolomic signature of AMI comprises or consists of 2, 3, 4, 5, 6, 7, 8, 9, 10, 11, 12, or 13 biomarkers selected from the group comprising or consisting of 1-monoolein, isomaltose, phenylalanine, L1PN, L1AB, L3TG, threose, H4A2, L1TG, TPA2, L1PL, glutamine, and glycerol.

In one embodiment, the metabolomic signature of AMI comprises at least 2, 3, 4, 5, 6, 7, 8, 9, 10, or 11 biomarkers selected from the group comprising or consisting of 1-monoolein, isomaltose, phenylalanine, L1PN, L1AB, L3TG, threose, H4A2, L1TG, TPA2, L1PL, and glutamine; and optionally further comprises glycerol. In one embodiment, the metabolomic signature of AMI comprises or consists of 2, 3, 4, 5, 6, 7, 8, 9, 10, 11, or 12 biomarkers selected from the group comprising or consisting of 1-monoolein, isomaltose, phenylalanine, L1PN, L1AB, L3TG, threose, H4A2, L1TG, TPA2, L1PL, and glutamine; and optionally further comprises glycerol.

In one embodiment, the metabolomic signature of AMI comprises at least or consists of 1-monoolein. In one embodiment, the metabolomic signature of AMI comprises at least or consists of 1-monoolein and isomaltose. In one embodiment, the metabolomic signature of AMI comprises at least or consists of 1-monoolein, isomaltose, and glutamine. In one embodiment, the metabolomic signature of AMI comprises at least or consists of 1-monoolein, isomaltose, glutamine, and phenylalanine. In one embodiment, the metabolomic signature of AMI comprises at least or consists of 1-monoolein, isomaltose, glutamine, phenylalanine, and glycerol. In one embodiment, the metabolomic signature of AMI comprises at least or consists of 1-monoolein, isomaltose, glutamine, phenylalanine, glycerol, and L1PN. In one embodiment, the metabolomic signature of AMI comprises at least or consists of 1-monoolein, isomaltose, glutamine, phenylalanine, glycerol, L1PN, and L1AB. In one embodiment, the metabolomic signature of AMI comprises at least or consists of 1-monoolein, isomaltose, glutamine, phenylalanine, glycerol, L1PN, L1AB, and L3TG. In one embodiment, the metabolomic signature of AMI comprises at least or consists of 1-monoolein, isomaltose, glutamine, phenylalanine, glycerol, L1PN, L1AB, L3TG, and threose. In one embodiment, the metabolomic signature of AMI comprises at least or consists of 1-monoolein, isomaltose, glutamine, phenylalanine, glycerol, L1PN, L1AB, L3TG, threose, and H4A2. In one embodiment, the metabolomic signature of AMI comprises at least or consists of 1-monoolein, isomaltose, glutamine, phenylalanine, glycerol, L1PN, L1AB, L3TG, threose, H4A2, and L1TG. In one embodiment, the metabolomic signature of AMI comprises at least or consists of 1-monoolein, isomaltose, glutamine, phenylalanine, glycerol, L1PN, L1AB, L3TG, threose, H4A2, L1TG, and TPA2. In one embodiment, the metabolomic signature of AMI comprises at least or consists of 1-monoolein, isomaltose, glutamine, phenylalanine, glycerol, L1PN, L1AB, L3TG, threose, H4A2, L1TG, TPA2, and L1PL. In one embodiment, the metabolomic signature of AMI comprises or consists of 1-monoolein, isomaltose, phenylalanine, L1PN, L1AB, L3TG, threose, H4A2, L1TG, TPA2, L1PL, glutamine, and glycerol.

In one embodiment, the metabolomic signature of AMI as described hereinabove further comprises at least 1 biomarker selected from the group comprising or consisting of melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid.

In one embodiment, the metabolomic signature of AMI comprises at least 1 or consists of 1 biomarker selected from the group comprising or consisting of 1-monoolein, isomaltose, phenylalanine, threose, H4A2, TPA2, glutamine, and glycerol. In one embodiment, the metabolomic signature of AMI comprises at least 1 or consists of 1 biomarker selected from the group comprising or consisting of 1-monoolein, isomaltose, phenylalanine, threose, H4A2, TPA2, and glutamine; and optionally further comprises glycerol. In one embodiment, the metabolomic signature of AMI comprises at least 1 or consists of 1 biomarker selected from the group comprising or consisting of 1-monoolein, isomaltose, phenylalanine, threose, H4A2, and TPA2; and optionally further comprises glycerol and/or glutamine.

In one embodiment, the metabolomic signature of AMI comprises at least 2, 3, 4, 5, 6, or 7 biomarkers selected from the group comprising or consisting of 1-monoolein, isomaltose, phenylalanine, threose, H4A2, TPA2, glutamine, and glycerol. In one embodiment, the metabolomic signature of AMI comprises or consists of 2, 3, 4, 5, 6, 7, or 8 biomarkers selected from the group comprising or consisting of 1-monoolein, isomaltose, phenylalanine, threose, H4A2, TPA2, glutamine, and glycerol. In one embodiment, the metabolomic signature of AMI comprises or consists of 1-monoolein, isomaltose, phenylalanine, threose, H4A2, TPA2, glutamine, and glycerol.

In one embodiment, the metabolomic signature of AMI comprises at least 2, 3, 4, 5, or 6 biomarkers selected from the group comprising or consisting of 1-monoolein, isomaltose, phenylalanine, threose, H4A2, TPA2, and glutamine; and optionally further comprises glycerol. In one embodiment, the metabolomic signature of AMI comprises or consists of 2, 3, 4, 5, 6, or 7 biomarkers selected from the group comprising or consisting of 1-monoolein, isomaltose, phenylalanine, threose, H4A2, TPA2, and glutamine; and optionally further comprises glycerol.

In one embodiment, the metabolomic signature of AMI comprises at least 1 or consists of 1 biomarker selected from the group comprising or consisting of isomaltose, glutamine, phenylalanine, glycerol, L1PN, and 1-monoolein. In one embodiment, the metabolomic signature of AMI comprises at least 1 or consists of 1 biomarker selected from the group comprising or consisting of isomaltose, glutamine, phenylalanine, L1PN, and 1-monoolein; and optionally further comprises glycerol. In one embodiment, the metabolomic signature of AMI comprises at least 1 or consists of 1 biomarker selected from the group comprising or consisting of isomaltose, phenylalanine, L1PN, and 1-monoolein; and optionally further comprises glycerol and/or glutamine.

In one embodiment, the metabolomic signature of AMI comprises at least 2, 3, 4, or 5 biomarkers selected from the group comprising or consisting of isomaltose, glutamine, phenylalanine, glycerol, L1PN, and 1-monoolein. In one embodiment, the metabolomic signature of AMI comprises or consists of 2, 3, 4, 5, or 6 biomarkers selected from the group comprising or consisting of isomaltose, glutamine, phenylalanine, glycerol, L1PN, and 1-monoolein.

In one embodiment, the metabolomic signature of AMI comprises at least 2, 3, or 4 biomarkers selected from the group comprising or consisting of isomaltose, glutamine, phenylalanine, L1PN, and 1-monoolein; and optionally further comprises glycerol. In one embodiment, the metabolomic signature of AMI comprises or consists of 2, 3, 4, or 5 biomarkers selected from the group comprising or consisting of isomaltose, glutamine, phenylalanine, L1PN, and 1-monoolein; and optionally further comprises glycerol.

In one embodiment, the metabolomic signature of AMI comprises or consists of isomaltose, glutamine, phenylalanine, glycerol, L1PN, and 1-monoolein.

In one embodiment, the metabolomic signature of AMI comprises at least 1 or consists of 1 biomarker selected from the group comprising or consisting of isomaltose, glutamine, phenylalanine, glycerol, and L1PN. In one embodiment, the metabolomic signature of AMI comprises at least 1 or consists of 1 biomarker selected from the group comprising or consisting of isomaltose, glutamine, phenylalanine, and L1PN; and optionally further comprises glycerol. In one embodiment, the metabolomic signature of AMI comprises at least 1 or consists of 1 biomarker selected from the group comprising or consisting of isomaltose, phenylalanine, and L1PN; and optionally further comprises glycerol and/or glutamine.

In one embodiment, the metabolomic signature of AMI comprises at least 2, 3, or 4 biomarkers selected from the group comprising or consisting of isomaltose, glutamine, phenylalanine, glycerol, and L1PN. In one embodiment, the metabolomic signature of AMI comprises or consists of 2, 3, 4, or 5 biomarkers selected from the group comprising or consisting of isomaltose, glutamine, phenylalanine, glycerol, and L1PN.

In one embodiment, the metabolomic signature of AMI comprises at least 2 or 3 biomarkers selected from the group comprising or consisting of isomaltose, glutamine, phenylalanine, and L1PN; and optionally further comprises glycerol. In one embodiment, the metabolomic signature of AMI comprises or consists of 2, 3, or 4 biomarkers selected from the group comprising or consisting of isomaltose, glutamine, phenylalanine, and L1PN; and optionally further comprises glycerol.

In one embodiment, the metabolomic signature of AMI comprises at least or consists of isomaltose. In one embodiment, the metabolomic signature of AMI comprises at least or consists of isomaltose and glutamine. In one embodiment, the metabolomic signature of AMI comprises at least or consists of isomaltose, glutamine, and phenylalanine. In one embodiment, the metabolomic signature of AMI comprises at least or consists of isomaltose, glutamine, phenylalanine, and glycerol. In one embodiment, the metabolomic signature of AMI comprises at least or consists of isomaltose, glutamine, phenylalanine, glycerol, and L1PN. In one embodiment, the metabolomic signature of AMI comprises at least or consists of isomaltose, glutamine, phenylalanine, glycerol, L1PN, and 1-monoolein. In one embodiment, the metabolomic signature of AMI comprises or consists of isomaltose, glutamine, phenylalanine, glycerol, and L1PN.

In one embodiment, the metabolomic signature of AMI as described hereinabove further comprises at least 1 biomarker selected from the group comprising or consisting of melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid.

In one embodiment, the level, amount, or concentration of a given biomarker of the metabolomic signature of AMI is considered characteristic of AMI when compared to a reference metabolomic signature.

In one embodiment, the reference metabolomic signature comprises or consists of the same biomarker(s) than that/those of the metabolomic signature of AMI as described hereinabove.

In one embodiment, the reference metabolomic signature comprises or consists of the same biomarker(s) than that/those of the metabolomic signature of AMI selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol.

In one embodiment, the reference metabolomic signature comprises or consists of the same biomarker(s) than that/those of the metabolomic signature of AMI selected from the group comprising or consisting of 1-monoolein, isomaltose, phenylalanine, L1PN, L1AB, L3TG, threose, H4A2, L1TG, TPA2, L1PL, glutamine, and glycerol. In one embodiment, the reference metabolomic signature comprises or consists of the same biomarker(s) than that/those of the metabolomic signature of AMI selected from the group comprising or consisting of 1-monoolein, isomaltose, phenylalanine, threose, H4A2, TPA2, glutamine, and glycerol. In one embodiment, the reference metabolomic signature comprises or consists of the same biomarker(s) than that/those of the metabolomic signature of AMI selected from the group comprising or consisting of isomaltose, glutamine, phenylalanine, glycerol, L1PN, and 1-monoolein. In one embodiment, the reference metabolomic signature comprises or consists of the same biomarker(s) than that/those of the metabolomic signature of AMI selected from the group comprising or consisting of isomaltose, glutamine, phenylalanine, glycerol, and L1PN.

In one embodiment, the reference metabolomic signature is derived from a reference subject or derived from a reference population of subjects.

In one embodiment, the reference subject is a mammal, preferably a primate. In one embodiment, the reference subject is a human.

In one embodiment, the reference population of subjects comprises at least or consists of 2, 3, 4, 5, 10, 15, 20, 25, 30, 35, 40, 45, 50, or more subjects.

In one embodiment, the reference metabolomic signature is derived from population studies, including, for example, subjects having a similar age range, subjects in the same or similar ethnic group, subjects having a chronic medical condition linked with an increased risk of AMI, or subjects with a history of mesenteric ischemia.

According to one embodiment, the reference metabolomic signature is derived from the level, amount, or concentration of the same biomarker(s) than that/those of the metabolomic signature of AMI measured in a biological sample from one or more subjects who are seeking medical attention for abdominal pain not caused by mesenteric ischemia (also referred to as non-ischemic abdominal pain). Thus, according to one embodiment, the reference metabolomic signature is derived from a reference population of subjects with non-ischemic abdominal pain.

According to one embodiment, the reference metabolomic signature is derived from the level, amount, or concentration of the same biomarker(s) than that/those of the metabolomic signature of AMI measured in a biological sample from one or more subjects who are substantially healthy. Thus, according to one embodiment, the reference metabolomic signature is derived from a reference population of subjects who are substantially healthy.

In one embodiment, a “substantially healthy subject” is a subject who has not been previously diagnosed or identified as suffering from AMI. In one embodiment, a “substantially healthy subject” is a subject who does not suffer from abdominal pain. In one embodiment, a “substantially healthy subject” is a subject who does not suffer from any known disease, disorder or condition. In one embodiment, a “substantially healthy subject” is a subject who is not seeking medical attention.

According to one embodiment, the reference metabolomic signature is derived from the level, amount, or concentration of the same biomarker(s) than that/those of the metabolomic signature of AMI measured in a biological sample from one or more subjects suffering from AMI. Thus, according to one embodiment, the reference metabolomic signature is derived from a reference population of subjects with AMI.

In one embodiment, the reference metabolomic signature is derived from statistical analyses and/or risk prediction data of a reference population of subjects as described hereinabove. In one embodiment, the reference metabolomic signature is derived from one or more algorithms. In one embodiment, the reference metabolomic signature is derived from a reference population of subjects as described hereinabove using a method of statistical and/or structural classification.

As mentioned above, in one embodiment, the metabolomic signature of the invention is characteristic of AMI when compared to a reference metabolomic signature. Accordingly, in one embodiment, “being characteristic of AMI” means that the level, amount, or concentration of biomarker(s) comprised within the metabolomic signature of the invention is substantially different from or substantially similar to the level, amount, or concentration of the same biomarker(s) measured in a biological sample from a reference subject or derived from a reference population of subjects.

In one embodiment, the metabolomic signature of the invention is characteristic of AMI if the level, amount, or concentration of at least 1, 2, 3, 4, 5, 6, 7, 8, 9, 10, 11, 12, 13, or more biomarker(s) comprised within the metabolomic signature of the invention is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects. In one embodiment, the metabolomic signature of the invention is characteristic of AMI if the level, amount, or concentration of 1, 2, 3, 4, 5, 6, 7, 8, 9, 10, 11, 12, 13, or more biomarker(s) comprised within the metabolomic signature of the invention is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects.

In one embodiment, the metabolomic signature of the invention is characteristic of AMI if the level, amount, or concentration of at least 1, 2, 3, 4, 5, 6, 7, 8, 9, 10, 11, 12, 13, or more biomarker(s) selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects. In one embodiment, the metabolomic signature of the invention is characteristic of AMI if the level, amount, or concentration of 1, 2, 3, 4, 5, 6, 7, 8, 9, 10, 11, 12, 13, or more biomarker(s) selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects.

In one embodiment, the metabolomic signature of the invention is characteristic of AMI if the level, amount, or concentration of at least 1, 2, 3, 4, 5, 6, 7, 8, 9, 10, 11, or 12 biomarker(s) selected from the group comprising or consisting of 1-monoolein, isomaltose, phenylalanine, L1PN, L1AB, L3TG, threose, H4A2, L1TG, TPA2, L1PL, glutamine, and glycerol, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects. In one embodiment, the metabolomic signature of the invention is characteristic of AMI if the level, amount, or concentration of 1, 2, 3, 4, 5, 6, 7, 8, 9, 10, 11, 12, or 13 biomarker(s) selected from the group comprising or consisting of 1-monoolein, isomaltose, phenylalanine, L1PN, L1AB, L3TG, threose, H4A2, L1TG, TPA2, L1PL, glutamine, and glycerol, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects.

In one embodiment, the metabolomic signature of the invention is characteristic of AMI if the level, amount, or concentration of at least 1, 2, 3, 4, or 5 biomarker(s) selected from the group comprising or consisting of isomaltose, glutamine, phenylalanine, glycerol, L1PN, and 1-monoolein, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects. In one embodiment, the metabolomic signature of the invention is characteristic of AMI if the level, amount, or concentration of 1, 2, 3, 4, 5, or 6 biomarker(s) selected from the group comprising or consisting of isomaltose, glutamine, phenylalanine, glycerol, L1PN, and 1-monoolein, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects.

In one embodiment, the metabolomic signature of the invention is characteristic of AMI if the level, amount, or concentration of at least 1, 2, 3, or 4 biomarker(s) selected from the group comprising or consisting of isomaltose, glutamine, phenylalanine, glycerol, and L1PN, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects. In one embodiment, the metabolomic signature of the invention is characteristic of AMI if the level, amount, or concentration of 1, 2, 3, 4, or 5 biomarker(s) selected from the group comprising or consisting of isomaltose, glutamine, phenylalanine, glycerol, and L1PN, is substantially different from the level, amount, or concentration of the same biomarker(s) measured in a biological sample from a reference subject either with non-ischemic abdominal pain or substantially healthy, or derived from a reference population either of subjects with non-ischemic abdominal pain or of substantially healthy subjects.

In one embodiment, the level, amount, or concentration of a given biomarker is “substantially different” if it is more than about 1%, 2%, 3%, 4%, 5%, 6%, 7%, 8%, 9%, 10%, 15%, 20%, 25%, 30%, 35%, 40%, 45%, or 50% higher or lower than the level, amount, or concentration of the same biomarker measured in the reference subject or derived from the reference population of subjects. In one embodiment, the level, amount, or concentration of a given biomarker is “substantially different” if it is more than about 1%, 2%, 3%, 4%, 5%, 10%, 15%, 20%, or 25% higher or lower than the level, amount, or concentration of the same biomarker measured in the reference subject or derived from the reference population of subjects. In one embodiment, the level, amount, or concentration of a given biomarker is “substantially different” if it is more than about 5% higher or lower than the level, amount, or concentration of the same biomarker measured in the reference subject or derived from the reference population of subjects.

In one embodiment, the metabolomic signature of the invention is characteristic of AMI if the level, amount, or concentration of at least 1, preferably at least 2, 3, 4, 5, 6, 7, 8, 9, 10, 11, 12, or all of the following biomarker(s) varies from the level, amount, or concentration of the same biomarker(s) measured in a biological sample from a reference subject with non-ischemic abdominal pain, or derived from a reference population of subjects with non-ischemic abdominal pain as indicated:
the level, amount, or concentration of 1-monoolein is substantially higher,
the level, amount, or concentration of isomaltose is substantially higher,
the level, amount, or concentration of phenylalanine is substantially higher,
the level, amount, or concentration of threose is substantially lower,
the level, amount, or concentration of glutamine is substantially lower,
the level, amount, or concentration of glycerol is substantially higher,
the level, amount, or concentration of H4A2 is substantially lower,
the level, amount, or concentration of TPA2 is substantially lower,
the level, amount, or concentration of L1PN is substantially higher,
the level, amount, or concentration of L1AB is substantially higher,
the level, amount, or concentration of L3TG is substantially higher,
the level, amount, or concentration of L1TG is substantially higher, and/or
the level, amount, or concentration of L1PL is substantially higher.

In one embodiment, the metabolomic signature of the invention is characteristic of AMI if the level, amount, or concentration of at least 1, preferably at least 2, 3, 4, 5, 6, 7, or all of the following biomarker(s) varies from the level, amount, or concentration of the same biomarker(s) measured in a biological sample from a reference subject with non-ischemic abdominal pain, or derived from a reference population of subjects with non-ischemic abdominal pain as indicated:
the level, amount, or concentration of 1-monoolein is substantially higher,
the level, amount, or concentration of isomaltose is substantially higher,
the level, amount, or concentration of phenylalanine is substantially higher,
the level, amount, or concentration of threose is substantially lower,
the level, amount, or concentration of glutamine is substantially lower,
the level, amount, or concentration of glycerol is substantially higher,
the level, amount, or concentration of H4A2 is substantially lower, and/or
the level, amount, or concentration of TPA2 is substantially lower.

In one embodiment, the metabolomic signature of the invention is characteristic of AMI if the level, amount, or concentration of at least 1, preferably at least 2, 3, 4, 5, or all of the following biomarker(s) varies from the level, amount, or concentration of the same biomarker(s) measured in a biological sample from a reference subject with non-ischemic abdominal pain, or derived from a reference population of subjects with non-ischemic abdominal pain as indicated:
the level, amount, or concentration of isomaltose is substantially higher,
the level, amount, or concentration of glutamine is substantially lower,
the level, amount, or concentration of phenylalanine is substantially higher,
the level, amount, or concentration of glycerol is substantially higher,
the level, amount, or concentration of L1PN is substantially higher, and/or
the level, amount, or concentration of 1-monoolein is substantially higher.

In one embodiment, the metabolomic signature of the invention is characteristic of AMI if the level, amount, or concentration of at least 1, preferably at least 2, 3, 4, or all of the following biomarker(s) varies from the level, amount, or concentration of the same biomarker(s) measured in a biological sample from a reference subject with non-ischemic abdominal pain, or derived from a reference population of subjects with non-ischemic abdominal pain as indicated:
the level, amount, or concentration of isomaltose is substantially higher,
the level, amount, or concentration of glutamine is substantially lower,
the level, amount, or concentration of phenylalanine is substantially higher,
the level, amount, or concentration of glycerol is substantially higher, and/or
the level, amount, or concentration of L1PN is substantially higher.

In one embodiment, the level, amount, or concentration of a given biomarker is substantially higher if it is more than about 1%, 2%, 3%, 4%, or 5% higher, preferably more than about 5% higher, than the level, amount, or concentration of the same biomarker measured in the reference subject or derived from the reference population of subjects. In one embodiment, the level, amount, or concentration of a given biomarker is substantially lower if it is more than about 1%, 2%, 3%, 4%, or 5% lower, preferably more than about 5% lower, than the level, amount, or concentration of the same biomarker measured in the reference subject or derived from the reference population of subjects.

Another object of the invention is a method for identifying a subject suffering or being at risk of suffering from acute mesenteric ischemia (AMI) by determining a metabolomic signature as described hereinabove. In one embodiment, the method is for identifying a subject suffering or being at risk of suffering from early AMI as described hereinabove.

According to one embodiment, the present invention relates to a method for identifying a subject suffering or being at risk of suffering from AMI, said method comprising or consisting of:
a) determining a metabolomic signature of the subject by measuring in a biological sample from the subject the level, amount, or concentration of biomarker(s) as described hereinabove;
b) comparing the metabolomic signature of the subject to a reference metabolomic signature; and
c) identifying a subject suffering or being at risk of suffering from AMI based on the comparison of his/her metabolomic signature to the reference metabolomic signature.

In one embodiment, “biological sample” refers to a bodily fluid, a cell, or a tissue or organ, in particular a tissue or organ biopsy. Examples of biological samples thus include whole blood, apheresis blood, serum, plasma, urine, feces, synovial fluid, bronchoalveolar lavage fluid, sputum, lymph, ascitic fluids, amniotic fluid, peritoneal fluid, cerebrospinal fluid, pleural fluid, pericardial fluid, and alveolar macrophages, tissue lysates, biopsies and extracts prepared from tissues.

In one embodiment, the biological sample from the subject is a bodily fluid sample. Examples of bodily fluid samples include a blood, plasma, serum, lymph, urine, or sweat sample.

In one embodiment, the biological sample from the subject is a blood sample. In one embodiment, the biological sample from the subject is a whole blood sample. In one embodiment, the biological sample from the subject is a plasma sample. Methods for obtaining a plasma sample are routinely used in clinical laboratories. In one embodiment, the whole blood sample or the plasma sample from the subject is processed to obtain a serum sample. Thus, in one embodiment, the biological sample from the subject is a serum sample. Methods for obtaining a serum sample from a whole blood sample or a plasma sample are routinely used in clinical laboratories.

According to one embodiment, the method of the invention comprises a step of obtaining or recovering a biological sample from the subject.

According to one embodiment, the method of the invention does not comprise a step of obtaining or recovering a biological sample from the subject. In said embodiment, the biological sample was previously obtained from the subject. Biological samples may be conserved in adequate conditions before being used in the method of the invention. Therefore, in one embodiment, the method of the invention is a non-invasive method or an in vitro method.

In one embodiment, the present invention relates to a method for identifying a subject suffering or being at risk of suffering from AMI, said method comprising or consisting of:
a) determining a metabolomic signature of the subject by measuring in a biological sample previously obtained from the subject the level, amount, or concentration of biomarker(s) as described hereinabove;
b) comparing the metabolomic signature of the subject to a reference metabolomic signature; and
c) identifying a subject suffering or being at risk of suffering from AMI based on the comparison of his/her metabolomic signature to the reference metabolomic signature.

According to one embodiment, determining a metabolomic signature of the subject means determining the presence of a metabolomic signature of AMI in the subject. Therefore, in one embodiment, determining a metabolomic signature of the subject comprises or consists of measuring in a biological sample from the subject the level, amount, or concentration of biomarker(s) comprised within the metabolomic signature of AMI as described hereinabove.

In one embodiment, determining a metabolomic signature of the subject comprises or consists of measuring in a biological sample from the subject the level, amount, or concentration of at least 1, 2, 3, 4, 5, 6, 7, 8, 9, 10, 11, 12, 13, or more biomarker(s) selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol. In one embodiment, determining a metabolomic signature of the subject comprises or consists of measuring in a biological sample from the subject the level, amount, or concentration of at least 1, 2, 3, 4, 5, 6, 7, 8, 9, 10, 11, 12, 13, or more biomarker(s) selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, and glutamine; and optionally further comprises measuring in a biological sample from the subject the level, amount, or concentration of glycerol.

In one embodiment, determining a metabolomic signature of the subject comprises or consists of measuring in a biological sample from the subject the level, amount, or concentration of at least 1, 2, 3, 4, 5, 6, 7, 8, 9, 10, 11, or 12, or all biomarker(s) selected from the group comprising or consisting of 1-monoolein, isomaltose, phenylalanine, L1PN, L1AB, L3TG, threose, H4A2, L1TG, TPA2, L1PL, glutamine, and glycerol. In one embodiment, determining a metabolomic signature of the subject comprises or consists of measuring in a biological sample from the subject the level, amount, or concentration of at least 1, 2, 3, 4, 5, 6, 7, 8, 9, 10, or 11, or all biomarker(s) selected from the group comprising or consisting of 1-monoolein, isomaltose, phenylalanine, L1PN, L1AB, L3TG, threose, H4A2, L1TG, TPA2, L1PL, and glutamine; and optionally further comprises measuring in a biological sample from the subject the level, amount, or concentration of glycerol.

In one embodiment, determining a metabolomic signature of the subject comprises or consists of measuring in a biological sample from the subject the level, amount, or concentration of at least 1, 2, 3, 4, or 5, or all biomarker(s) selected from the group comprising or consisting of isomaltose, glutamine, phenylalanine, glycerol, L1PN, and 1-monoolein. In one embodiment, determining a metabolomic signature of the subject comprises or consists of measuring in a biological sample from the subject the level, amount, or concentration of at least 1, 2, 3, or 4, or all biomarker(s) selected from the group comprising or consisting of isomaltose, glutamine, phenylalanine, glycerol, and L1PN.

Therefore, in one embodiment, the present invention relates to a method for identifying a subject suffering or being at risk of suffering from AMI, said method comprising or consisting of:
a) measuring in a biological sample from the subject the level, amount, or concentration of biomarker(s) as described hereinabove;
b) comparing the level, amount, or concentration of biomarker(s) measured in a biological sample from the subject to the level, amount, or concentration of the same biomarker(s) measured in a biological sample from a reference subject as described hereinabove or derived from a reference population of subjects as described hereinabove; and
c) identifying a subject suffering or being at risk of suffering from AMI based on said comparison.

In one embodiment, step a) of the method comprises or consists of measuring in a biological sample from the subject the level, amount, or concentration of at least 1, 2, 3, 4, 5, 6, 7, 8, 9, 10, 11, 12, 13, or more biomarker(s) selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol. In one embodiment, step a) of the method comprises or consists of measuring in a biological sample from the subject the level, amount, or concentration of at least 1, 2, 3, 4, 5, 6, 7, 8, 9, 10, 11, 12, 13, or more biomarker(s) selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, and glutamine; and optionally further comprises measuring in a biological sample from the subject the level, amount, or concentration of glycerol.

In one embodiment, step a) of the method comprises or consists of measuring in a biological sample from the subject the level, amount, or concentration of at least 1, 2, 3, 4, 5, 6, 7, 8, 9, 10, 11, or 12, or all biomarker(s) selected from the group comprising or consisting of 1-monoolein, isomaltose, phenylalanine, L1PN, L1AB, L3TG, threose, H4A2, L1TG, TPA2, L1PL, glutamine, and glycerol. In one embodiment, step a) of the method comprises or consists of measuring in a biological sample from the subject the level, amount, or concentration of at least 1, 2, 3, 4, 5, 6, 7, 8, 9, 10, or 11, or all biomarker(s) selected from the group comprising or consisting of 1-monoolein, isomaltose, phenylalanine, L1PN, L1AB, L3TG, threose, H4A2, L1TG, TPA2, L1PL, and glutamine; and optionally further comprises measuring in a biological sample from the subject the level, amount, or concentration of glycerol.

In one embodiment, step a) of the method comprises or consists of measuring in a biological sample from the subject the level, amount, or concentration of at least 1, 2, 3, 4, or 5, or all biomarker(s) selected from the group comprising or consisting of isomaltose, glutamine, phenylalanine, glycerol, L1PN, and 1-monoolein. In one embodiment, step a) of the method comprises or consists of measuring in a biological sample from the subject the level, amount, or concentration of at least 1, 2, 3, or 4, or all biomarker(s) selected from the group comprising or consisting of isomaltose, glutamine, phenylalanine, glycerol, and L1PN.

According to one embodiment, step b) of the method comprises or consists of comparing the metabolomic signature determined for the subject to a reference metabolomic signature as described hereinabove.

In one embodiment, the reference metabolomic signature comprises or consists of the same biomarker(s) than that/those comprised within the metabolomic signature determined for the subject.

In one embodiment, the reference metabolomic signature is derived from a reference subject or from a reference population of subjects, preferably from a reference subject with non-ischemic abdominal pain or from a reference population of subjects with non-ischemic abdominal pain.

In one embodiment, step b) of comparing the metabolomic signature determined for the subject to a reference metabolomic signature is computer-implemented. Thus, in one embodiment, the method for identifying a subject suffering or being at risk of suffering from acute mesenteric ischemia (AMI) is computer-implemented.

According to one embodiment, step c) of the method comprises or consists of identifying a subject suffering or being at risk of suffering from AMI based on the comparison of the metabolomic signature determined for said subject to a reference metabolomic signature as described hereinabove.

In one embodiment, the subject is identified as suffering or being at risk of suffering from AMI if the metabolomic signature determined for said subject is substantially different from a reference metabolomic signature, in particular from a reference metabolomic signature derived from a reference subject with non-ischemic abdominal pain or from a reference population of subjects with non-ischemic abdominal pain.

In one embodiment, the subject is identified as not suffering or not being at risk of suffering from AMI if the metabolomic signature determined for said subject is substantially similar to a reference metabolomic signature, in particular to a reference metabolomic signature derived from a reference subject with non-ischemic abdominal pain or from a reference population of subjects with non-ischemic abdominal pain.

In one embodiment, the subject is identified as suffering or being at risk of suffering from AMI if the level, amount, or concentration of at least 1, 2, 3, 4, 5, 6, 7, 8, 9, 10, 11, 12, 13, or more biomarker(s) comprised within the metabolomic signature determined for said subject is substantially different from the level, amount, or concentration of the same biomarker(s) comprised within the reference metabolomic signature.

In one embodiment, the subject is identified as suffering or being at risk of suffering from AMI if the level, amount, or concentration of at least 1, 2, 3, 4, 5, 6, 7, 8, 9, 10, 11, 12, 13, or more biomarker(s) selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, acetic acid, glutamine, and glycerol, is substantially different from the level, amount, or concentration of the same biomarker(s) comprised within the reference metabolomic signature.

In one embodiment, the subject is identified as suffering or being at risk of suffering from AMI if the level, amount, or concentration of at least 1, 2, 3, 4, 5, 6, 7, 8, 9, 10, 11, or 12, or all biomarker(s) selected from the group comprising or consisting of 1-monoolein, isomaltose, phenylalanine, L1PN, L1AB, L3TG, threose, H4A2, L1TG, TPA2, L1PL, glutamine, and glycerol, is substantially different from the level, amount, or concentration of the same biomarker(s) comprised within the reference metabolomic signature.

In one embodiment, the subject is identified as suffering or being at risk of suffering from AMI if the level, amount, or concentration of at least 1, 2, 3, 4, or 5, or all biomarker(s) selected from the group comprising or consisting of isomaltose, glutamine, phenylalanine, glycerol, L1PN, and 1-monoolein, and is substantially different from the level, amount, or concentration of the same biomarker(s) comprised within the reference metabolomic signature. In one embodiment, the subject is identified as suffering or being at risk of suffering from AMI if the level, amount, or concentration of at least 1, 2, 3, or 4, or all biomarker(s) selected from the group comprising or consisting of isomaltose, glutamine, phenylalanine, glycerol, and L1PN, and is substantially different from the level, amount, or concentration of the same biomarker(s) comprised within the reference metabolomic signature.

In one embodiment, the level, amount, or concentration of a given biomarker is “substantially different” if it is more than about 1%, 2%, 3%, 4%, 5%, 6%, 7%, 8%, 9%, 10%, 15%, 20%, 25%, 30%, 35%, 40%, 45%, or 50% higher or lower than the level, amount, or concentration of the same biomarker comprised within the reference metabolomic signature. In one embodiment, the level, amount, or concentration of a given biomarker is “substantially different” if it is more than about 1%, 2%, 3%, 4%, 5%, 10%, 15%, 20%, or 25% higher or lower than the level, amount, or concentration of the same biomarker comprised within the reference metabolomic signature. In one embodiment, the level, amount, or concentration of a given biomarker is “substantially different” if it is more than about 5% higher or lower than the level, amount, or concentration of the same biomarker comprised within the reference metabolomic signature.

In one embodiment, the subject is identified as suffering or being at risk of suffering from AMI if the level, amount, or concentration of at least 1, preferably at least 2, 3, 4, 5, 6, 7, 8, 9, 10, 11, 12, or all of the following biomarker(s) varies from the level, amount, or concentration of the same biomarker(s) comprised within the reference metabolomic signature derived from a reference subject with non-ischemic abdominal pain, or derived from a reference population of subjects with non-ischemic abdominal pain, as indicated:
the level, amount, or concentration of 1-monoolein is substantially higher,
the level, amount, or concentration of isomaltose is substantially higher,
the level, amount, or concentration of phenylalanine is substantially higher,
the level, amount, or concentration of threose is substantially lower,
the level, amount, or concentration of glutamine is substantially lower,
the level, amount, or concentration of glycerol is substantially higher,
the level, amount, or concentration of H4A2 is substantially lower,
the level, amount, or concentration of TPA2 is substantially lower,
the level, amount, or concentration of L1PN is substantially higher,
the level, amount, or concentration of L1AB is substantially higher,
the level, amount, or concentration of L3TG is substantially higher,
the level, amount, or concentration of L1TG is substantially higher, and/or
the level, amount, or concentration of L1PL is substantially higher.

In one embodiment, the level, amount, or concentration of a given biomarker is substantially higher if it is more than about 1%, 2%, 3%, 4%, or 5% higher, preferably more than about 5% higher, than the level, amount, or concentration of the same biomarker measured in the reference subject or derived from the reference population of subjects. In one embodiment, the level, amount, or concentration of a given biomarker is substantially lower if it is more than about 1%, 2%, 3%, 4%, or 5% lower, preferably more than about 5% lower, than the level, amount, or concentration of the same biomarker measured in the reference subject or derived from the reference population of subjects.

Another object of the invention is a method for providing an adapted care to a subject identified as suffering or being at risk of suffering from acute mesenteric ischemia (AMI). In one embodiment, the method is for providing an adapted care to a subject identified as suffering or being at risk of suffering from early AMI as described hereinabove.

According to one embodiment, the present invention relates to a method for providing an adapted care to a subject identified as suffering or being at risk of suffering from AMI, said method comprising or consisting of:
identifying a subject as suffering or being at risk of suffering from AMI as described hereinabove; and
providing an adapted care to the subject identified as suffering or being at risk of suffering from AMI.

Thus, in one embodiment, the present invention relates to a method for providing an adapted care to a subject identified as suffering or being at risk of suffering from AMI,
said method comprising or consisting of:
a) determining a metabolomic signature of the subject by measuring in a biological sample from the subject the level, amount, or concentration of biomarker(s) as described hereinabove;
b) comparing the metabolomic signature of the subject to a reference metabolomic signature as described hereinabove; and
c) providing an adapted care to the subject identified as suffering or being at risk of suffering from AMI based on the comparison of his/her metabolomic signature to the reference metabolomic signature as described hereinabove.

In one embodiment, providing an adapted care to the subject identified as suffering or being at risk of suffering from AMI comprises at least one or consists of one of the following:
performing an abdominal computed tomography (CT) scan, preferably an abdominal CT angiogram (CTA), more preferably an abdominal contrast-enhanced CTA, on said subject identified as suffering or being at risk of suffering from AMI;
monitoring the subject identified as suffering or being at risk of suffering from AMI, in particular for risk factors of necrosis;
providing urgent medical therapy; and/or
providing an urgent interventional treatment.

In one embodiment, providing an adapted care to the subject identified as suffering or being at risk of suffering from AMI comprises or consists of performing an abdominal CT scan, preferably an abdominal CT angiogram (CTA), more preferably an abdominal contrast-enhanced CTA, on said subject identified as suffering or being at risk of suffering from AMI, in particular to confirm the diagnosis and/or guide an interventional treatment (such as vascular and/or digestive surgery).

In one embodiment, providing an adapted care to the subject identified as suffering or being at risk of suffering from AMI comprises or consists of monitoring the subject identified as suffering or being at risk of suffering from AMI. In one embodiment, providing an adapted care to the subject identified as suffering or being at risk of suffering from AMI comprises or consists of monitoring the subject identified as suffering or being at risk of suffering from AMI for risk factors of necrosis. Examples of risk factors of necrosis in a subject identified as suffering or being at risk of suffering from AMI include, without being limited to, an elevated serum lactate concentration, organ failure(s), and small bowel dilatation. In one embodiment, monitoring the subject identified as suffering or being at risk of suffering from AMI for risk factors of necrosis comprises at least one or consists of one of the following:
monitoring the serum lactate concentration of the subject identified as suffering or being at risk of suffering from AMI;
monitoring the presence of organ failure(s) in the subject identified as suffering or being at risk of suffering from AMI;
monitoring the presence of small bowel dilatation in the subject identified as suffering or being at risk of suffering from AMI.

In one embodiment, an elevated serum lactate concentration is a serum lactate concentration higher than about 2 mmol/L. Methods for measuring serum lactate concentration are well-known in the art and are routinely used in clinical laboratories.

In one embodiment, organ failure(s) (sometimes also known as organ dysfunction(s)) comprises at least one of: respiratory failure (or respiratory dysfunction), cardiovascular failure (or cardiovascular dysfunction), liver failure (or liver dysfunction), renal failure (renal dysfunction), and neurological failure (neurological dysfunction). Methods for assessing the presence of organ failure(s) or organ dysfunction(s) are well-known in the art and include, for example, assessing the presence of organ failure or organ dysfunction with a score, such as the sequential organ failure assessment (SOFA) score.

In one embodiment, the presence of small bowel dilatation is defined as the presence of a dilatation of the small bowel of at least about 2.5 cm. Methods for assessing the presence of small bowel dilatation are well-known in the art and include, for example, performing an abdominal CT scan.

In one embodiment, providing an adapted care to the subject identified as suffering or being at risk of suffering from AMI comprises or consists of providing urgent medical therapy. Examples of urgent medical therapy include, without being limited to, the administration of antithrombotics and/or antibiotics.

In one embodiment, providing an adapted care to the subject identified as suffering or being at risk of suffering from AMI comprises or consists of providing an urgent interventional treatment. Examples of urgent medical interventional treatment, without being limited to, vascular and/or digestive surgery.

In one embodiment, the present invention relates to a method for providing an adapted care to a subject identified as suffering or being at risk of suffering from AMI, said method comprising or consisting of:
identifying a subject as suffering or being at risk of suffering from AMI as described hereinabove; and
providing an adapted care to the subject identified as suffering or being at risk of suffering from AMI comprising or consisting of performing an abdominal CT scan, preferably an abdominal CT angiogram (CTA), more preferably an abdominal contrast-enhanced CTA, on the subject identified as suffering or being at risk of suffering from AMI.

Another object of the invention is a method for treating a subject identified as suffering or being at risk of suffering from acute mesenteric ischemia (AMI). In one embodiment, the method is for treating a subject identified as suffering or being at risk of suffering from early AMI as described hereinabove.

According to one embodiment, the present invention relates to a method for treating a subject identified as suffering or being at risk of suffering from AMI, said method comprising or consisting of:
identifying a subject as suffering or being at risk of suffering from AMI as described hereinabove; and
administering a treatment to the subject identified as suffering or being at risk of suffering from AMI.

In one embodiment, the present invention relates to a method for treating a subject identified as suffering or being at risk of suffering from AMI, said method comprising or consisting of:
a) determining a metabolomic signature of the subject by measuring in a biological sample from the subject the level, amount, or concentration of biomarker(s) as described hereinabove;
b) comparing the metabolomic signature of the subject to a reference metabolomic signature as described hereinabove; and
c) administering a treatment to the subject identified as suffering or being at risk of suffering from AMI based on the comparison of his/her metabolomic signature to the reference metabolomic signature as described hereinabove.

According to one embodiment, administering a treatment to the subject identified as suffering or being at risk of suffering from AMI comprises or consists of providing an adapted care as described hereinabove.

According to one embodiment, administering a treatment to the subject identified as suffering or being at risk of suffering from AMI comprises at least one or consists of one of the following:
performing fluid resuscitation;
administering oral antibiotics such as, for example, gentamicin and/or metronidazole;
administering proton pump inhibitors such as, for example, pantoprazole;
administering antithrombotics such as, for example, unfractionated heparin;
administering oxygen therapy; and/or
performing revascularization, such as endovascular or surgical revascularization.

In one embodiment, the present invention relates to a method for treating a subject identified as suffering or being at risk of suffering from AMI, said method comprising or consisting of:
identifying a subject as suffering or being at risk of suffering from AMI as described hereinabove; and
treating the subject identified as suffering or being at risk of suffering from AMI by performing fluid resuscitation; by administering at least one of: oral antibiotics, proton pump inhibitors, antithrombotics, and/or oxygen therapy; and/or by performing revascularization.

In one embodiment, the subject is a mammal, preferably a primate. In one embodiment, the subject is a human.

In one embodiment, the subject is a patient, i.e., a mammal, preferably a primate, more preferably a human, who/which is awaiting the receipt of, or is receiving medical attention or was/is/will be the object of a medical procedure, or is monitored for the development of a disease.

In one embodiment, the subject is a human with abdominal pain. In one embodiment, the subject is a human seeking medical attention for abdominal pain. In one embodiment, the subject is a human admitted to the emergency room with abdominal pain.

In one embodiment, the subject is a male. In one embodiment, the subject is a female. In one embodiment, the subject is older than 50, 55, 60, 65, 70, 75, 80, or 85 years of age.

In one embodiment, the subject has a history of cardiovascular disease. In one embodiment, the subject is overweight. In one embodiment, the subject has a BMI (body mass index) higher than about 25 kg/m².

Another object of the invention is a kit comprising or consisting of means for determining the metabolomic signature of AMI as described hereinabove and for implementing the method for identifying a subject suffering or being at risk of suffering from AMI as described hereinabove.

According to one embodiment, the kit of the invention comprises or consists of:
means for measuring the level, amount, or concentration of biomarker(s) comprised within the metabolomic signature of AMI as described hereinabove;
optionally internal standard(s) and/or control(s); and
optionally instructions for use.

According to one embodiment, the kit of the invention comprises or consists of:
means for measuring the level, amount, or concentration of biomarker(s) comprised within the metabolomic signature of AMI as described hereinabove;
internal standard(s) and/or control(s); and
instructions for use.

In one embodiment, the kit of the invention comprises or consists of means for measuring the level, amount, or concentration of at least 1 biomarker selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid, glutamine, and glycerol. In one embodiment, the kit of the invention comprises or consists of means for measuring the level, amount, or concentration of at least 1 biomarker selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid, and glutamine; and optionally further comprises means for measuring the level, amount, or concentration of glycerol. In one embodiment, the kit of the invention comprises or consists of means for measuring the level, amount, or concentration of at least 1 biomarker selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid; and optionally further comprises means for measuring the level, amount, or concentration of glycerol and/or glutamine.

In one embodiment, the kit of the invention comprises or consists of means for measuring the level, amount, or concentration of at least 2, 3, 4, 5, 6, 7, 8, 9, 10, 11, 12, 13, or more biomarkers selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid, glutamine, and glycerol. In one embodiment, the kit of the invention comprises or consists of means for measuring the level, amount, or concentration of at least 2, 3, 4, 5, 6, 7, 8, 9, 10, 11, 12, 13, or more biomarkers selected from the group comprising or consisting of 1-monoolein, isomaltose, phenylalanine, L1PN, L1AB, L3TG, threose, H4A2, L1TG, TPA2, L1PL, melibiose, alpha-tocopherol, 2-hydroxybutyric acid, D-allose, 2-ethylhexanoic acid, threonic acid, palmitic acid, succinic acid, oleic acid, quinic acid, pyruvic acid, phosphoric acid, cholesterol, 2-ketoisocaproic acid, glyceric acid, H4PL, HDA2, H4A1, H4CH, H3FC, H4FC, HDA1, TPA1, L6FC, L5PL, L5CH, H3A2, L5FC, H3PL, L5AB, L5PN, L6PL, and acetic acid, and glutamine; and optionally further comprises means for measuring the level, amount, or concentration of glycerol.

In one embodiment, the kit of the invention comprises or consists of means for measuring the level, amount, or concentration of at least 1 biomarker selected from the group comprising or consisting of 1-monoolein, isomaltose, phenylalanine, L1PN, L1AB, L3TG, threose, H4A2, L1TG, TPA2, L1PL, glutamine, and glycerol. In one embodiment, the kit of the invention comprises or consists of means for measuring the level, amount, or concentration of at least 1 biomarker selected from the group comprising or consisting of 1-monoolein, isomaltose, phenylalanine, L1PN, L1AB, L3TG, threose, H4A2, L1TG, TPA2, L1PL, and glutamine; and optionally further comprises means for measuring the level, amount, or concentration of glycerol. In one embodiment, the kit of the invention comprises or consists of means for measuring the level, amount, or concentration of at least 1 biomarker selected from the group comprising or consisting of 1-monoolein, isomaltose, phenylalanine, L1PN, L1AB, L3TG, threose, H4A2, L1TG, TPA2, and L1PL; and optionally further comprises means for measuring the level, amount, or concentration of glycerol and/or glutamine.

In one embodiment, the kit of the invention comprises or consists of means for measuring the level, amount, or concentration of at least 2, 3, 4, 5, 6, 7, 8, 9, 10, 11, or 12, or all biomarkers selected from the group comprising or consisting of 1-monoolein, isomaltose, phenylalanine, L1PN, L1AB, L3TG, threose, H4A2, L1TG, TPA2, L1PL, glutamine, and glycerol. In one embodiment, the kit of the invention comprises or consists of means for measuring the level, amount, or concentration of at least 2, 3, 4, 5, 6, 7, 8, 9, 10, 11, or all biomarkers selected from the group comprising or consisting of 1-monoolein, isomaltose, phenylalanine, L1PN, L1AB, L3TG, threose, H4A2, L1TG, TPA2, L1PL, and glutamine; and optionally further comprises means for measuring the level, amount, or concentration of glycerol.

In one embodiment, the kit of the invention comprises or consists of means for measuring the level, amount, or concentration of at least 1 biomarker selected from the group comprising or consisting of isomaltose, glutamine, phenylalanine, glycerol, L1PN, and 1-monoolein. In one embodiment, the kit of the invention comprises or consists of means for measuring the level, amount, or concentration of at least 1 biomarker selected from the group comprising or consisting of isomaltose, glutamine, phenylalanine, L1PN, and 1-monoolein; and optionally further comprises means for measuring the level, amount, or concentration of glycerol. In one embodiment, the kit of the invention comprises or consists of means for measuring the level, amount, or concentration of at least 1 biomarker selected from the group comprising or consisting of isomaltose, phenylalanine, L1PN, and 1-monoolein; and optionally further comprises means for measuring the level, amount, or concentration of glycerol and/or glutamine.

In one embodiment, the kit of the invention comprises or consists of means for measuring the level, amount, or concentration of at least 2, 3, 4, 5, or all biomarkers selected from the group comprising or consisting of isomaltose, glutamine, phenylalanine, glycerol, L1PN, and 1-monoolein. In one embodiment, the kit of the invention comprises or consists of means for measuring the level, amount, or concentration of at least 2, 3, 4, or all biomarkers selected from the group comprising or consisting of isomaltose, glutamine, phenylalanine, L1PN, and 1-monoolein; and optionally further comprises means for measuring the level, amount, or concentration of glycerol.

In one embodiment, the kit of the invention comprises or consists of means for measuring the level, amount, or concentration of at least 1 biomarker selected from the group comprising or consisting of isomaltose, glutamine, phenylalanine, glycerol, and L1PN. In one embodiment, the kit of the invention comprises or consists of means for measuring the level, amount, or concentration of at least 1 biomarker selected from the group comprising or consisting of isomaltose, glutamine, phenylalanine, and L1PN; and optionally further comprises means for measuring the level, amount, or concentration of glycerol. In one embodiment, the kit of the invention comprises or consists of means for measuring the level, amount, or concentration of at least 1 biomarker selected from the group comprising or consisting of isomaltose, phenylalanine, and L1PN; and optionally further comprises means for measuring the level, amount, or concentration of glycerol and/or glutamine.

In one embodiment, the kit of the invention comprises or consists of means for measuring the level, amount, or concentration of at least 2, 3, 4, or all biomarkers selected from the group comprising or consisting of isomaltose, glutamine, phenylalanine, glycerol, and L1PN. In one embodiment, the kit of the invention comprises or consists of means for measuring the level, amount, or concentration of at least 2, 3, or all biomarkers selected from the group comprising or consisting of isomaltose, glutamine, phenylalanine, glycerol, and L1PN; and optionally further comprises means for measuring the level, amount, or concentration of glycerol.

In one embodiment, the kit of the invention comprises or consists of means for measuring the level, amount, or concentration of 1-monoolein, isomaltose, phenylalanine, L1PN, L1AB, L3TG, threose, H4A2, L1TG, TPA2, L1PL, glutamine, and glycerol. In one embodiment, the kit of the invention comprises or consists of means for measuring the level, amount, or concentration of 1-monoolein, isomaltose, phenylalanine, threose, H4A2, TPA2, glutamine, and glycerol. In one embodiment, the kit of the invention comprises or consists of means for measuring the level, amount, or concentration of isomaltose, glutamine, phenylalanine, glycerol, L1PN, and 1-monoolein. In one embodiment, the kit of the invention comprises or consists of means for measuring the level, amount, or concentration of isomaltose, glutamine, phenylalanine, glycerol, and L1PN.

Another object of the invention is the use of reagents for determining the metabolomic signature of AMI as described hereinabove in the manufacture of a diagnostic agent or a kit for performing a method for identifying a subject suffering or being at risk of suffering from AMI as described hereinabove.

### BRIEF DESCRIPTION OF THE DRAWINGS

FIG. 1 is a flowchart illustrating the screening and selection of the AMI patients and non-AMI patients (controls) included in the study. Abbreviations: AMI: acute mesenteric ischemia; CT scan: contrast-enhanced computed tomography scan; CMI: chronic mesenteric ischemia; GC-MS: gas chromatography-mass spectrometry; ¹H-NMR: hydrogen-1 nuclear magnetic resonance spectroscopy also known as proton nuclear magnetic resonance spectrometry; PCA: principal component analysis.

FIGS. 2A-M are a set of box-plots showing acute mesenteric ischemia related plasma metabolites identified in the study. 0=control patients; 1=patients with acute mesenteric ischemia. P-values are adjusted on age and sex, and corrected for false discovery rate (Benjamini-Hochberg). The horizontal line in the boxes represents the median, and the bottom and top of the boxes the 25th and 75th percentiles, respectively. I bars represent the upper adjacent value (75th percentile plus 1.5 times the interquartile range) and the lower adjacent value (corresponding formula below the 25th percentile), and the dots/asterisks represent outliers.

FIG. 3 is a graph showing the 13-metabolite model accuracies according to the number of metabolites on LAMDA integrative analysis applied on a training set (85% of the cohort). Best AUC/number of features ratio was obtained with the following 13 metabolites (and their corresponding weight): 1-monoolein (1.00), isomaltose (0.72), glutamine (0.66), phenylalanine (0.57), glycerol (0.57), L1PN (0.56), L1AB (0.56), L3TG (0.55), threose (0.55), H4A2 (0.53), L1TG (0.53), TPA2 (0.51), L1PL (0.50).

FIG. 4 is a graph showing the 5-metabolite model accuracies according to the number of metabolites on LAMDA integrative analysis applied on a training set (85% of the cohort), excluding the 1-monoolein biomarker. Best AUC/number of features ratio was obtained with the following 5 metabolites (and their corresponding weight): isomaltose (1.00), glutamine (0.92), phenylalanine (0.80), glycerol (0.80), L1PN (0.79).

FIGS. 5A-C are a set of box-plots showing the concentrations of 1-monoolein, isomaltose and glutamine in control patients, patients with early acute mesenteric ischemia (early AMI) and patients with late necrotic acute mesenteric ischemia (necrotic AMI). P-values are adjusted on age and sex, and corrected for false discovery rate (Benjamini-Hochberg). The horizontal line in the boxes represents the median, and the bottom and top of the boxes, the 25^{th} and 75^{th} percentiles, respectively. I bars represent the upper adjacent value (75^{th} percentile plus 1.5 times the interquartile range) and the lower adjacent value (corresponding formula below the 25^{th} percentile), and the dots/asterisks outliers. n=34 patients with early acute mesenteric ischemia and n=13 patients with late necrotic early acute mesenteric ischemia.

### EXAMPLES

The present invention is further illustrated by the following examples.

### Example: Cross-Sectional Study from an Intestinal Stroke Center

### Materials and Methods

### Study Design and Setting

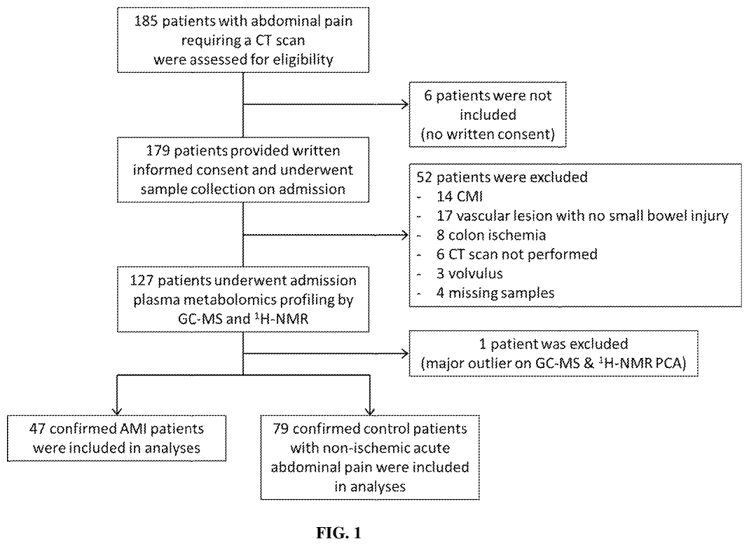

The study was a cross-sectional diagnostic study enrolling patients with acute abdominal pain requiring a contrast-enhanced computed tomography (CT) scan from January 2016 to March 2018. Patients with acute mesenteric ischemia (AMI) were admitted to the intestinal stroke center (Beaujon Hospital, Clichy, France), whereas patients in whom an AMI diagnosis was ruled out (controls) were admitted to the emergency room (see patient flowchart, FIG. 1). Blood samples were collected from all the patients on admission and stored for further biomarker analysis. The study was approved by the Ethics Committee of Paris-Nord Val de Seine University Hospitals. The study complies with the Standards for Reporting Diagnostic Accuracy (STARD) guidelines (Bossuyt et al., BMJ. 2015 Oct. 28; 351:h5527). Written informed consent was obtained from all patients.

### Patients and Controls

AMI was defined by the association of 1) acute clinical, biological and/or contrast-enhanced CT scan features of bowel injury, 2) vascular insufficiency (occlusive or non-occlusive) of the celiac trunk and/or the superior mesentery artery and/or superior mesenteric vein, and 3) the absence of an alternative diagnosis (Nuzzo et al., Am J Gastroenterol. 2019 February; 114(2):348-351). The diagnosis was confirmed by histology following intestinal resection (Nuzzo et al., Am J Gastroenterol. 2017 April; 112(4):597-605). All the AMI patients were managed by a standardized multimodal and multidisciplinary approach, as previously described (Corcos et al., Clin Gastroenterol Hepatol. 2013 February; 11(2):158-65.e2). Briefly, the patients were systematically administered oral antibiotics and antithrombotics, and emergency endovascular revascularization of arterial AMI was performed whenever technically feasible. Alternatively, open surgical revascularization was performed. Bowel viability was evaluated by laparotomy, decided based on published risk factors for irreversible transmural intestinal necrosis: occurrence of organ failure, elevated serum lactate concentrations, small bowel dilatation or perforation on CT scan (Nuzzo et al., Am J Gastroenterol. 2017 April; 112(4):597-605). Irreversible transmural intestinal necrosis was confirmed upon pathological assessment.

The diagnosis of AMI was ruled in or out by the CT scan, and alternative final diagnoses were based on clinical, biologic, and CT scan findings. Finally, all the included patients underwent a contrast-enhanced abdominal CT scan, routine biologic work-up, and blood and urine sampling. Any patient presenting with a diagnosis of left-sided colon ischemia, chronic mesenteric ischemia without acute injury, vascular lesions with no intestinal injury, or strangulated bowel obstruction was excluded (see patient flowchart, FIG. 1). All the patients' clinical records, CT scans, and pathologic specimens were reviewed in a multidisciplinary meeting including gastroenterologists, radiologists, digestive and vascular surgeons and intensivists, all experts in digestive vascular diseases, to avoid diagnostic misclassification before any blood sample analyses.

### Data Collection and Processing

Routine baseline clinical and biological characteristics were collected upon admission for all patients: age, gender, body mass index (BMI), history of cardiovascular disease or risk factors, general and digestive clinical signs and common biologic features. The origin of AMI (arterial—thrombotic or embolic—venous, or non-occlusive) was specified based on the patient's records, CT scan, and pathologic review. Plasma samples were collected in heparin-treated tubes before immediate centrifugation at 3,000 rpm for 15 min at room temperature and subsequent storage at −80° C. until further analysis. Metabolomic analyses were performed after the final diagnosis classification had been made in all patients.

### Metabolomics Spectra Acquisition

Analyses were performed at Imperial College London, using validated GC-MS (gas chromatography-mass spectrometry) and ¹H-NMR (proton nuclear magnetic resonance spectrometry). Sample preparation and GC-MS spectra acquisition were performed as follows. A protein/lipid precipitation protocol was optimized to effectively clean-up the samples while minimizing the alterations of the metabolome. Furthermore, the analytes were derivatized under a dual-stage procedure (methoxamine (MOX) followed by N-methyl-trimethylsilyl-trifluoroacetamide (MSTFA)) to make them analyzable by GC-MS and maximize metabolome coverage. Metabolite annotation was performed by comparing the spectra and retention times with the Fiehn library (Kind et al., Anal Chem. 2009 Dec. 15; 81(24):10038-48) and the NIST (National Institute of Standards and Technology) library. Subsequently, the annotated peaks were visually inspected one by one to correct the missed annotations based on the biological relevance of the annotated metabolites, their physicochemical properties and the choice of analytical platform.

Peak picking was performed with Gavin3 package in Matlab to ensure that all the targeted metabolites were accurately integrated. Reproducibility, blank contamination, instrument drift, run order and batch effects were evaluated with the use of supervised and unsupervised multivariate statistics and corrected accordingly, with the aim to control and minimize the analytical variability and focus exclusively in the biological variability.

Methods for sample preparation and ¹H-NMR spectra acquisition on a Bruker spectrometer (Rheinstetten, Germany) operating at 600.22 MHz frequency were as described previously (Dona et al., Anal Chem. 2014 Oct. 7; 86(19):9887-94; Dumas et al., Nat Genet. 2007 May; 39(5):666-72).

### Statistical Analyses

### Metadata

For each of the continuous variables, the median and the interquartile range (IQR) were reported. Categorical variables were expressed as the number of observations and percentages. Normally distributed quantitative data were analyzed with the Student t-test. Mann-Whitney U test was used otherwise. Qualitative data were compared with either the Pearson χ² test or the Fisher exact test, depending on the sample size. It was determined that the enrollment of 50 patients in each group would provide a power of more than 95% for assessing diagnostic tests with AUC≥0.70 (Obuchowski et al., Clin Chem. 2004 July; 50(7):1118-25).

### Metabolomics

Unsupervised principal component analysis (PCA) models, which do not include knowledge of the results of the reference standard, were produced to investigate whether there were any hard outliers, due to either analytical error or biological deviation. PCA is a multivariate projection method, used to extract and display systematic variation in a data matrix. The score plots of the PCA models display correlations between the participants metabolic profiles, with points closer together representing more similar profiles, allowing groups and trends to be revealed.

Metabolomics data analysis pertains to all metabolites including amino acids, carbohydrates, lipids, nucleotides, microbiota metabolism, energy, cofactors and vitamins, xenobiotics, and novel metabolites. To identify biochemicals that differed significantly between groups, metabolites were compared by univariate and multivariate logistic regression adjusting for age and sex. An estimate of the false discovery rate (FDR) was calculated to consider the multiple comparisons that normally occur in metabolomic-based studies. The univariate and multivariate tables were ranked according to p-values and q-values (false discovery rates adjustment). Then a machine learning analysis (fuzzy feature selection algorithm—MEMBAS (MEmbership Margin Based Attribute Selection)) was performed to select panels of diagnostic biomarkers with the best AUC/number of metabolites ratio. The algorithm was applied on a training set of 85% of patients and a validation set of 15% of patients.

### Fuzzy Feature Selection Algorithm (MEMBAS)

Fuzzy set theory was proposed by Zadeh in 1965 to mathematically model the imprecision inherent to some concepts. In short, fuzzy set theory allows an object to partially (simultaneously) belong to a set (class) with a certain degree of membership between 0 and 1. In a machine learning framework, an approach is defined as “fuzzy” if it is considered that an individual belongs to each class with a certain degree of membership, unlike the “crisp” (“hard”) approaches where each individual is considered to belong only to one class.

Existing feature selection algorithms are traditionally characterized as wrappers or filters according to the criterion used to search the relevant features. The selection algorithm referred to as MEMBAS (for MEmbership Margin Based Attribute Selection) enables to process similarly the three data types (numerical, qualitative, interval) based on an appropriate mapping using fuzzy logic concepts. The algorithm measures simultaneously the contribution of each metabolite for each of the two classes (patients and controls), in order to find the best discriminations. That is, it extracts the most pertinent markers since it is based on feature weighting according to the maximization of a membership margin. To avoid the heuristic search during the feature selection procedure, MEMBAS optimizes a membership margin based objective function by using classical optimization techniques providing an analytical solution.

### Class Prediction—Fuzzy Classification Algorithm

The learning and classification algorithm, LAMDA (Learning Algorithm for Multivariable Data Analysis) has been used to generate the fuzzy partitions that best discriminate AMI patients and abdominal pain control patients according to their metabolomic profiles. LAMDA is a fuzzy methodology of conceptual clustering and classification which is based on finding the global membership degree of a sample to an existing class, considering all the contributions of each feature. These contributions are called the marginal adequacy degrees (MADs). The MADs are calculated by means of a membership function and are then combined using “fuzzy mixed connectives” as aggregation operators in order to obtain the global adequacy degree (GAD) of an element to a class. Finally, a sample (AMI patient/control patient) will be assigned to the class for which its GAD is the highest. In Hedjazi et al. (Hedjazi et al., J Comput Biol. 2013 August; 20(8):610-20), and in the corresponding PhD thesis (Lyamine Hedjazi, Outil d'aide au diagnostic du cancer d partir d'extraction d'informations issues de bases de donnies et d'analyses par biopuces. Automatic Control Engineering. Université Paul Sabatier—Toulouse III, 2011), an extensive experimental study, including a comparison with known feature selection methods has been performed on several datasets presenting mixed-type and high-dimensional data. The experimental results in these works show that MEMBAS leads to a significant improvement of classification performance of LAMDA (fuzzy classifier) as well as other well-known classifiers (k-NN, SVM). Moreover, the combined fuzzy model MEMBAS/LAMDA works well in datasets with mixed-type data, since the same fuzzifying process (membership functions) is used for both feature selection and classification. This provides a similar processing for each feature type with minimal loss of information.

The diagnostic values of the panels of biomarkers identified were evaluated by analyzing the receiver operating curve (ROC) with the calculation of the area under the ROC (AUROC). All tests were two-sided. No imputation of missing data was performed. Analyses were performed using the Statistical Package for the Social Sciences (SPSS) for Mac OSX software (version 23.0, Chicago, IL, USA) and in R software, version 3.6.2 (R Foundation for Statistical Computing).

### Results

### Characteristics of Study Subjects

Between Jan. 4, 2016, and Mar. 5, 2018, 185 patients with acute abdominal pain requiring a contrast-enhanced CT scan were assessed for eligibility. Contrast-enhanced abdominal CT scan was performed and blood samples collected from 173 patients, including 77 admitted to the intestinal stroke center (Beaujon Hospital, Clichy, France) for suspicion of AMI and 96 admitted to the emergency room for non-ischemic abdominal pain (see Flowchart, FIG. 1). 44 patients were excluded from analysis based on the final diagnosis, or missing samples, which left 129 patients included—50 confirmed AMI and 79 non-AMI patients (controls). The baseline characteristics of both populations are summarized in Table 1 below.

TABLE 1
Baseline characteristics of AMI patients and controls.

| | AMI patients | | control patients | | |
|---|---|---|---|---|---|
| | n = 47 (%) | | n = 79 (%) | | p-value |
| Age, years* | 65 | (55-75) | 45 | (35-71) | <0.001 |
| BMI, kg/m²* | 27 | (21-33) | 22 | (20-24) | <0.001 |
| Female | 19 | (40) | 31 | (39) | 0.89 |

Atherosclerosis risk factors

| | | | | | |
|---|---|---|---|---|---|
| Tobacco use | 23 | (49) | 16 | (20) | 0.002 |
| Arterial hypertension | 28 | (59) | 20 | (25) | <0.001 |
| Dyslipidemia | 19 | (40) | 12 | (15) | 0.003 |
| Diabetes mellitus | 12 | (26) | 5 | (6) | 0.004 |

Cardiovascular history

| | | | | | |
|---|---|---|---|---|---|
| Myocardial ischemia | 9 | (19) | 5 | (6) | 0.04 |
| Stroke | 6 | (13) | 5 | (6) | 0.34 |
| Limb ischemia | 9 | (19) | 2 | (3) | 0.003 |
| Atrial fibrillation | 11 | (23) | 3 | (4) | 0.001 |
| Deep vein thrombosis | 3 | (6) | 4 | (5) | 1.00 |
| Pulmonary embolism | 5 | (11) | 4 | (5) | 0.31 |

Other comorbidities

| | | | | | |
|---|---|---|---|---|---|
| Chronic kidney disease | 1 | (2) | 2 | (3) | 1.00 |
| Cirrhosis | 4 | (9) | 4 | (5) | 0.71 |
| Abdominal surgery | 27 | (57) | 37 | (47) | 0.43 |

Clinical features

| | | | | | |
|---|---|---|---|---|---|
| Temperature* | 37.0 | (36.3-37.1) | 36.8 | (36.5-37.5) | 0.49 |
| Mean arterial pressure* | 100.3 | (90.8-110.3) | 96.0 | (83.7-105.7) | 0.17 |
| Vomiting | 21 | (44) | 39 | (49) | 0.41 |
| Diarrhea | 12 | (26) | 13 | (17) | 0.29 |
| Hematochezia | 8 | (17) | 3 | (4) | 0.02 |
| Guarding | 16 | (34) | 13 | (17) | 0.04 |
| Organ dysfunction | 15 | (32) | 8 | (10) | 0.004 |
| (total SOFA ≥ 2) | | | | | |

Abbreviations:
AMI: acute mesenteric ischemia;
BMI: body mass index;
SOFA: sequential organ failure assessment;
ASAT: aspartate aminotransferase
*median (interquartile range)

The final diagnosis of the controls is presented in Table 2 below. Patients with AMI (median age: 65 years (55-75), 38% of women) included arterial and venous causes in respectively 66% and 34% of cases. None of the included patients had non-occlusive AMI. AMI occurred in seven patients exhibiting signs of chronic mesenteric ischemia. Patients with AMI were significantly older, had a higher BMI, and were more likely to have risk factors or a history of cardiovascular disease than controls (see Table 1). AMI patients were also more likely to present hematochezia, guarding, and organ dysfunction (as measured by a total SOFA (sequential organ failure assessment) score ≥2) and a higher white blood cell count at baseline. Other baselines clinical and laboratory characteristics, including L-lactate, did not differ significantly (see Table 1). After admission to the intestinal stroke center (Beaujon Hospital, Clichy, France), AMI patients received antiplatelet therapy (n=33, 100% of arterial AMI patients), anticoagulants (n=50, 100% of AMI patients), oral antibiotics (n=49, 98% of AMI patients), and intravenous antibiotics (n=21, 42% of AMI patients). Emergency revascularization was performed in 29 patients (88% of arterial AMI patients).

TABLE 2
Etiological diagnosis of the control
group with acute abdominal pain.

| Diagnosis | n = 79 (%) |
|---|---|
| Infectious | 18 (23) |
| Diverticulitis | 9 |
| Appendicitis | 4 |
| Other causes of peritonitis/abdominal abscess | 5 |
| Inflammatory | 15 (19) |
| Intra-abdominal neoplasm progression | 8 |
| IBD flares | 7 |
| Bowel obstruction (non-strangulated) | 13 (16) |
| Functional G.I. disorders (reflux, diarrhea, constipation . . .) | 12 (15) |
| Biliopancreatic tract | 10 (13) |
| Pancreatitis | 7 |
| Biliary complications | 3 |
| Urogenital | 10 (13) |
| Invasive meningococcal disease | 1 (1) |

Abbreviations:
IBD: inflammatory bowel disease;
G.I.: gastro-intestinal

### Metabolomics

218 different metabolites were identified, including 97 and 127 through GC-MS and ¹H-NMR profiling, respectively. Some metabolites, such as glycerol, were identified through both GC-MS and ¹H-NMR profiling. The metabolites the most positively and negatively associated with a diagnosis of AMI in logistic regression (adjusted for age and sex) are shown in Table 3 below.

TABLE 3
Acute mesenteric ischemia related plasma metabolites based
on multivariate logistic regression (age and sex adjusted)

| | | | |
|---|---|---|---|
| Compound | Estimates | p-values | adjusted p-values |

GC-MS metabolites

| | | | |
|---|---|---|---|
| 1-monoolein | 4.70389 | 1.33e−06 | 1.08e−04 |
| melibiose | 1.824187 | 1.68e−05 | 4.54e−04 |
| alpha-tocopherol | −2.5361 | 1.41e−05 | 4.54e−04 |
| isomaltose | 1.640267 | 3.98e−05 | 8.06e−04 |
| 2-hydroxybutyric acid | 1.818458 | 6.24e−05 | 1.01e−03 |
| threose | −2.50963 | 0.000140173 | 1.80e−03 |
| D-allose | 1.669573 | 0.000155207 | 1.80e−03 |
| 2-ethylhexanoic acid | −1.70034 | 0.000560675 | 5.68e−03 |
| threonic acid | −1.20665 | 0.001608285 | 1.45e−02 |
| palmitic acid | −1.667887 | 0.001943953 | 1.57e−02 |
| succinic acid | 2.02738 | 0.003143423 | 2.12e−02 |
| oleic acid | 1.273331 | 0.002962736 | 2.12e−02 |
| quinic acid | −0.62265 | 0.003841036 | 2.39e−02 |
| pyruvic acid | −1.23496 | 0.00472627 | 2.56e−02 |
| phosphoric acid | −1.9044 | 0.004748942 | 2.56e−02 |
| glycerol | 1.735394 | 0.005133062 | 2.60e−02 |
| cholesterol | −2.83442 | 0.005787982 | 2.76e−02 |
| 2-ketoisocaproic acid | −1.56142 | 0.006613241 | 2.86e−02 |
| glyceric acid | −1.40387 | 0.009348605 | 3.79e−02 |

¹H-NMR metabolites

| | | | |
|---|---|---|---|
| glutamine | −7.42315 | 1.75e−05 | 6.67e−04 |
| glycerol | 6.66567 | 1.36e−05 | 6.67e−04 |
| H4A2 | −0.1968 | 2.56e−05 | 6.67e−04 |
| H4PL | −0.16603 | 2.90e−05 | 6.67e−04 |
| HDA2 | −0.1898 | 2.28e−05 | 6.67e−04 |
| TPA2 | −0.17824 | 1.45e−05 | 6.67e−04 |
| H4A1 | −0.05513 | 5.03e−05 | 9.75e−04 |
| H4CH | −0.16939 | 5.65e−05 | 9.75e−04 |
| H3FC | −1.11921 | 0.000184702 | 2.83e−03 |
| H4FC | −0.6547 | 0.000296495 | 3.72e−03 |
| HDA1 | −0.02764 | 0.000475334 | 5.05e−03 |
| TPA1 | −0.02888 | 0.000442939 | 5.05e−03 |
| L6FC | −0.31397 | 0.000637088 | 6.28e−03 |
| L5PL | −0.19109 | 0.00069921 | 6.43e−03 |
| L5CH | −0.10191 | 0.000781915 | 6.74e−03 |
| H3A2 | −0.58864 | 0.000967893 | 7.81e−03 |
| L5FC | −0.36079 | 0.001018216 | 7.81e−03 |
| H3PL | −0.26557 | 0.001592942 | 1.06e−02 |
| L5AB | −0.13923 | 0.001620641 | 1.06e−02 |
| L5PN | −0.00766 | 0.001618221 | 1.06e−02 |
| L6PL | −0.15294 | 0.001870843 | 1.17e−02 |
| acetic acid | 18.27703 | 0.002741432 | 1.40e−02 |
| phenylalanine | 19.97602 | 0.002342867 | 1.40e−02 |

A machine learning algorithm (fuzzy feature selection algorithm) including the 218 identified metabolites was implemented in order to identify the metabolites, and panels thereof, that could best discriminate patients with AMI from control patients.

A panel of 13 metabolites (glutamine, phenylalanine, isomaltose, threose, 1-monoolein, TPA2, L3TG, glycerol, L1PN, L1TG, H4A2, L1AB, and L1PL) discriminating patients with AMI from control patients was thus selected by fuzzy logic machine learning algorithm applied on a training set (85% of the cohort). 8 of the 13 metabolites (glutamine, phenylalanine, isomaltose, threose, 1-monoolein, TPA2, glycerol, and H4A2) are among the metabolites the most positively and negatively associated with a diagnosis of AMI in logistic regression presented in Table 3.

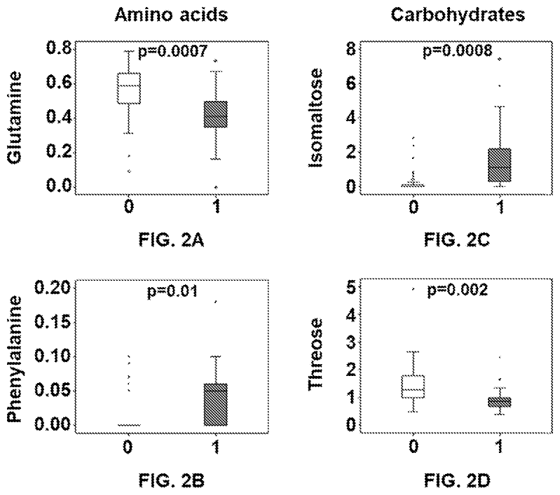

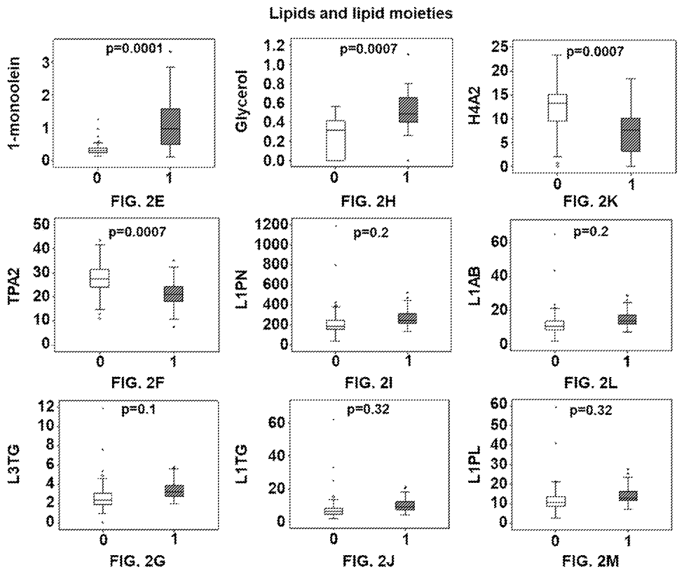

Individual comparisons for each of the 13 metabolites show that the levels of glutamine (FIG. 2A), threose (FIG. 2D), TPA2 (FIG. 2F) and H4A2 (FIG. 2K) were lower in AMI patients than in control patients, while the levels of phenylalanine (FIG. 2B), isomaltose (FIG. 2C), 1-monoolein (FIG. 2E), L3TG (FIG. 2G), glycerol (FIG. 2H), L1PN (FIG. 2I), L1TG (FIG. 2J), L1AB (FIG. 2L) and L1PL (FIG. 2M) were higher in AMI patients than in control patients.

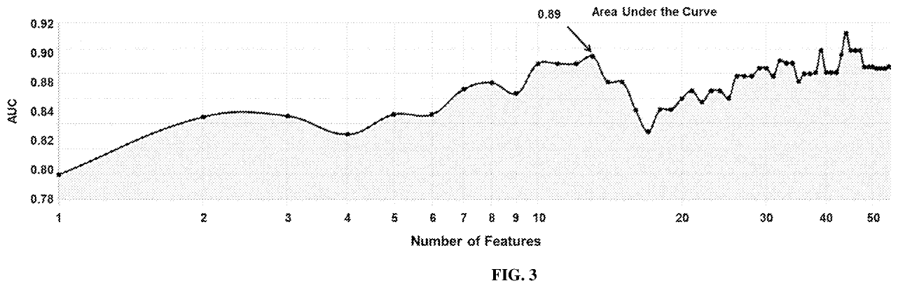

The combination of 13 metabolites allowed the identification of patients with AMI with an AUC=0.89 in the training set (85% of the cohort) (FIG. 3), indicating an excellent diagnostic performance. After internal validation (cross-validation) performed on the 15% of the cohort (18 patients) corresponding to the validation set, all cases of AMI were identified with an error rate of 0% (AUC=1.00). The unsupervised validation of the results on the data reduced to 13 biomarkers by principal component analysis and hierarchical clustering shows a clear separation of the two groups of patients (data not shown).

Furthermore, FIG. 3 shows that a lesser number of metabolites can actually be used to identify AMI patients with a very good diagnostic performance. For example, 1-monoolein alone allowed the identification of patients with AMI with an AUC=0.8 (as determined on a training set corresponding to 85% of the cohort).

The individual AUROC (also known as AUC) of each of the 13 metabolites identified by LAMDA integrative analysis are presented in Table 4. Said individual AUROC were calculated on the whole cohort. In particular, 1-monoolein, isomaltose and glycerol have an AUROC superior to 0.8, indicative of an excellent diagnostic performance. In addition, glutamine, L1PN, L1AB, L3TG, threose, H4A2, L1TG, TPA2, phenylalanine, and L1PL have an AUROC superior to 0.7 or very close to 0.7, indicative of a satisfactory diagnostic performance.

TABLE 4
AUROC of the 13 metabolites with the best AUC/number
of features ratio by LAMDA integrative analysis

| Metabolites | AUROC (95% CI)* |
|---|---|
| 1-monoolein | 0.858 (0.782-0.933) |
| Isomaltose | 0.848 (0.770-0.927) |
| Glutamine | 0.781 (0.698-0.863) |
| Phenylalanine | 0.681 (0.580-0.782) |
| Glycerol | 0.817 (0.736-0.897) |
| L1PN | 0.729 (0.642-0.816) |
| L1AB | 0.729 (0.642-0.816) |
| L3TG | 0.752 (0.668-0.835) |
| Threose | 0.789 (0.709-0.870) |
| H4A2 | 0.776 (0.692-0.861) |
| L1TG | 0.741 (0.654-0.828) |
| TPA2 | 0.792 (0.709-0.875) |
| L1PL | 0.698 (0.607-0.789) |

*AUROC calculated on the whole cohort for every biomarker individually

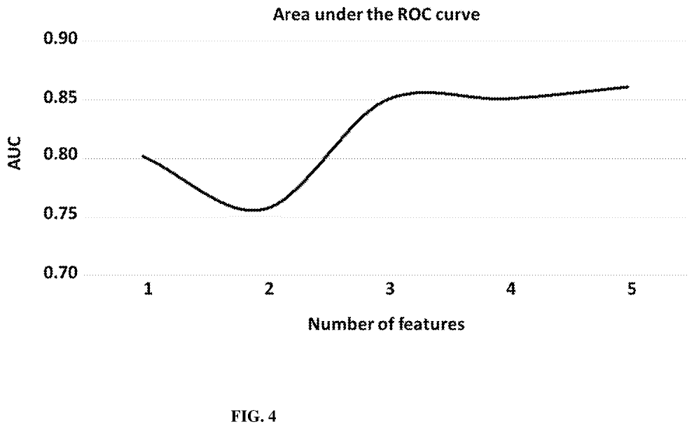

In a secondary model excluding the 1-monoolein biomarker, the fuzzy logic machine learning algorithm applied on the training set (85% of the cohort) selected a panel of 5 metabolites (glutamine, phenylalanine, isomaltose, glycerol, L1PN) discriminating patients with AMI from control patients with an AUC=0.86 (FIG. 4), indicating an excellent diagnostic performance. On the validation set (15% of the cohort, n=18), this combination of 5 metabolites identified AMI patients with an AUC=0.93.

As noted above, FIG. 4 thus confirms that a lesser number of metabolites can actually be used to identify AMI patients with a very good diagnostic performance. For example, isomaltose alone allowed the identification of patients with AMI with an AUC of about 0.8 (as determined on a training set corresponding to 85% of the cohort).

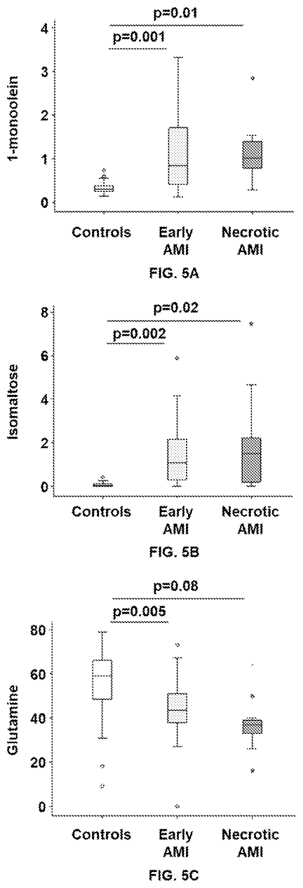

Finally, the concentrations of 1-monoolein, isomaltose and glutamine were compared between abdominal pain control patients, patients with early acute mesenteric ischemia (early AMI) and patients with late necrotic acute mesenteric ischemia (necrotic AMI) (FIG. 5A-C). As shown by the significant difference between control patients and early AMI patients, each marker individually was powerful enough to allow discrimination between early AMI patients and abdominal pain control patients.

In conclusion, the data demonstrate that the metabolite biomarkers identified herein, in particular glutamine, phenylalanine, isomaltose, threose, 1-monoolein, TPA2, L3TG, glycerol, L1PN, L1TG, H4A2, L1AB, and L1PL, can be measured, either alone or in combinations, in order to identify patients suffering or being at risk of suffering from acute mesenteric ischemia (AMI).

1-15. (canceled)

16. A method for providing an adapted care to a subject identified as suffering or being at risk of suffering from acute mesenteric ischemia (AMI), said method comprising:
a) determining a metabolomic signature of the subject by measuring in a biological sample previously obtained from the subject the level, amount, or concentration of at least one biomarker selected from the group consisting of isomaltose, glutamine, phenylalanine, glycerol, L1PN, 1-monoolein, L1AB, L3TG, threose, H4A2, L1TG, TPA2, and L1PL;
b) comparing the metabolomic signature of the subject to a reference metabolomic signature; and
c) providing an adapted care to the subject identified as suffering or being at risk of suffering from AMI based on the comparison of his/her metabolomic signature to the reference metabolomic signature.

17. A method for identifying a subject suffering or being at risk of suffering from acute mesenteric ischemia (AMI), said method comprising:
a) determining a metabolomic signature of the subject by measuring in a biological sample previously obtained from the subject the level, amount, or concentration of at least one biomarker selected from the group consisting of isomaltose, glutamine, phenylalanine, glycerol, L1PN, 1-monoolein, L1AB, L3TG, threose, H4A2, L1TG, TPA2, and L1PL;
b) comparing the metabolomic signature of the subject to a reference metabolomic signature; and
c) identifying the subject suffering or being at risk of suffering from AMI based on the comparison of his/her metabolomic signature to the reference metabolomic signature.

18. The method according to claim 16, wherein the metabolomic signature is determined by measuring in a biological sample previously obtained from the subject the level, amount, or concentration of at least one biomarker selected from the group consisting of isomaltose, glutamine, phenylalanine, glycerol, L1PN, and 1-monoolein.

19. The method according to claim 16, wherein the metabolomic signature is determined by measuring in a biological sample previously obtained from the subject the level, amount, or concentration of isomaltose, glutamine, phenylalanine, glycerol and L1PN.

20. The method according to claim 16, wherein the metabolomic signature is determined by measuring in a biological sample previously obtained from the subject the level, amount, or concentration of isomaltose, glutamine, phenylalanine, glycerol, L1PN, 1-monoolein, L1AB, L3TG, threose, H4A2, L1TG, TPA2, and L1PL.

21. The method according to claim 16, wherein the reference metabolomic signature is derived from the measure of the level, amount, or concentration of the same at least one biomarker in biological samples previously obtained from a population of subjects suffering from non-ischemic abdominal pain.

22. The method according to claim 16, wherein the biological sample previously obtained from the subject is a blood sample.

23. The method according to claim 16, wherein the biological sample previously obtained from the subject is a plasma sample.

24. A kit for implementing the method according to claim 16, wherein said kit comprises:
means for measuring the level, amount, or concentration of at least one biomarker selected from the group consisting of isomaltose, glutamine, phenylalanine, glycerol, L1PN, 1-monoolein, L1AB, L3TG, threose, H4A2, L1TG, TPA2, and L1PL;
optionally internal standard(s) and/or control(s); and
optionally instructions for use.

25. The kit according to claim 24, wherein said kit comprises means for measuring the level, amount, or concentration of at least one biomarker selected from the group consisting of isomaltose, glutamine, phenylalanine, glycerol, L1PN, and 1-monoolein.

26. The kit according to claim 24, wherein said kit comprises means for measuring the level, amount, or concentration of isomaltose, glutamine, phenylalanine, glycerol and L1PN.

27. The kit according to claim 24, wherein said kit comprises means for measuring the level, amount or concentration of isomaltose, glutamine, phenylalanine, glycerol, L1PN, 1-monoolein, L1AB, L3TG, threose, H4A2, L1TG, TPA2, and L1PL.

28. A metabolomic signature of acute mesenteric ischemia (AMI), said metabolomic signature comprising at least one biomarker selected from the group consisting of isomaltose, glutamine, phenylalanine, glycerol, L1PN, 1-monoolein, L1AB, L3TG, threose, H4A2, L1TG, TPA2, and L1PL.

29. The metabolomic signature of AMI according to claim 28, wherein said metabolomic signature comprises at least one biomarker selected from the group consisting of isomaltose, glutamine, phenylalanine, glycerol, L1PN, and 1-monoolein.

30. The metabolomic signature of AMI according to claim 28, wherein said metabolomic signature comprises isomaltose, glutamine, phenylalanine, glycerol, and L1PN.

31. The metabolomic signature of AMI according to claim 28, wherein said metabolomic signature comprises isomaltose, glutamine, phenylalanine, glycerol, L1PN, 1-monoolein, L1AB, L3TG, threose, H4A2, L1TG, TPA2, and L1PL.
